SEQUENCE LISTING (3 sequences)

SEQ ID NO: 1 - DNA, 1523 bp, Streptomyces bottropensis
  source 1..1523: mol_type = genomic DNA
acggagagtttgatcctggctcaggacgaacgctggcggcgtgcttaacacatgcaagtcgaacgatgaaccacttcggtggggattagtggcgaacgggtgagtaacacgtgggcaatctgccctgcactctgggacaagccctggaaacggggtctaataccggataacacacgctcaggcatctgagtgtgttgaaagctccggcggtgcaggatgagcccgcggcctatcagcttgttggtgaggtagaagctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggttgtgaaagcccggggcttaaccccgggtctgcagtcgatacgggcaggctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcatcagagatggtgccccccttgtggtcggtatacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggcaggtacaatgagctgcgaagccgcgaggcggagcgaatctcaaaaagcctgtctcagttcggattggggtctgcaactcgaccccatgaagtcggagttgctagtaatcgcagatcagcattgctgcggtgaatacgttcccgggccttgtacacaccgcccgtcacgtcacgaaagtcggtaacacccgaagccggtggcccaaccccttgtgggagggagctgtcgaaggtgggactggcgattgggacgaagtcgtaacaaggtagccgtaccggaaggtgcggctggatcacctccttt

SEQ ID NO: 2 - DNA, 20 bp, Synthetic construct
  source 1..20: mol_type = other DNA
aaactyaaakgaattgacgg

SEQ ID NO: 3 - DNA, 16 bp, synthetic construct
  source 1..16: mol_type = other DNA
gttgctcgcgttggga

(19) United States
(12) Patent Application Publication
(10) Pub. No.: US 2026/0206769 A1
(43) Pub. Date: Jul. 23, 2026

(54) Products and Methods for Pathogen Control In Plants

(71) Applicants: Aphea.Bio NV, Zwijnaarde (BE); Fundación Centro De Excelencia En Investigación De Medicamentos Innovadores En Andalucía, Medina, Granada (ES)

(72) Inventors: Thomas Simon, Zwijnaaarde (BE); Michael Houbraken, Lokeren (BE); Thijs De Mulder, Drongen (BE); Steven Vandenabeele, Oudenaarde (BE); Isabel Vercauteren, Woubrechtegem (BE); Emma Lagae, Gent (BE); Jens Maintz, Zwijnaarde (BE)

(21) Appl. No.: 19/144,118

(22) PCT Filed: Dec. 22, 2023
(86) PCT No.: PCT/EP2023/087579

§ 371 Date: Jun. 27, 2025

(30) Foreign Application Priority Data
Dec. 28, 2022 (EP) 22383307.0

(51) Int. Cl. A01N 63/28; A01P 3/00; C12N 1/205; C12R 1/465

(52) U.S. Cl. CPC A01N 63/28; A01P 3/00; C12N 1/205; C12R 2001/465

(57) ABSTRACT

The application concerns methods for conferring a pathogen control or resistance in a plant using certain bacteria or agricultural active compositions comprising the bacteria. Plants and plant parts treated with or heterologously disposed with said bacteria or compositions are also disclosed. Further, novel bacterial strains, and populations and agricultural active compositions comprising the same are provided.

### CROSS REFERENCE TO RELATED APPLICATIONS

This application is a national phase entry under 35 U.S.C. § 371 of International Patent Application PCT/EP2023/087579, filed Dec. 22, 2023, designating the United States of America and published in English as International Patent Publication WO2024/141453 on Jul. 4, 2024, which claims the benefit under Article 8 of the Patent Cooperation Treaty to European Patent Application Serial No. 22383307.0, filed Dec. 28, 2022, the entireties of which are hereby incorporated by reference.

### INCORPORATION BY REFERENCE

The ST.26 XML Sequence listing named “11015-10963-US2025-06-06-SequenceListingST6.xml”, created on Dec. 15, 2023, and having a size of 8,192 bytes, is hereby incorporated herein by this reference in its entirety.

### FIELD OF THE INVENTION

The invention is broadly in the field of plant biology and bacterial strains, more precisely in the field of pathogen control and resistance using bacteria. In particular, the invention relates to products and methods for controlling these pathogens.

### BACKGROUND OF THE INVENTION

There is a continuous increase in the demand for agricultural products, in order to be able to feed the growing human population as well as the cattle. In addition, such growing demand puts a further strain on the environment, because of the conventional practices used in agriculture. The most serious problem encountered in the cultivation of crops is the loss of material and damage caused by harmful and often destructive plant pathogens.

Various methods have been developed so far to control plant disease. Among them, the most commonly used and the most developed method is the chemical control method using chemical pesticides. Chemical pesticides are convenient to use and have immediate effects to protect plants from pests, but are listed as specified poisonous substances, deleterious substance, etc., which are regulated by law. In recent years, the abuse of chemical pesticides has created social problems: intoxications and deaths caused by acute toxicity; contamination of food due to residual pesticides in agricultural products; and influence of the outflow of residual pesticides on the human body and environment. Furthermore, new pests resistant to previous chemical pesticides are emerging, forcing the development of new types of pesticides, creating an endless cycle.

A promising practice is the use of microorganisms for battling and controlling plant diseases, and there is much need in the market for bio-based pathogen control methods and means, preferably broad-spectrum methods that are not harmful for the environment and the consumer.

### SUMMARY OF THE INVENTION

The present invention is at least in part based on the inventors' discovery that certain bacteria can be used for pathogen control or resistance in plants, thereby reducing disease parameters such as the disease severity and the presence of necrosis in the plants.

As corroborated in the experimental section, which illustrates certain representative embodiments of the invention, the present inventors have found inter alia that plants treated with the bacteria demonstrated a decrease in disease severity and/or a decrease in development of necrosis caused by a variety of pathogens, as compared to untreated plants.

Accordingly, an aspect of the invention relates to a method for conferring a pathogen control or resistance in a plant, the method comprising administering bacteria of a bacterial strain which comprises a 16S polynucleotide having 100% sequence identity to SEQ ID NO: 1 (see Table 2) to the plant, a part thereof, a seed for growing the plant, or a locus of the plant.

A related aspect provides a method for conferring a pathogen control or resistance in a plant, the method comprising administering a microbial active ingredient that is derived from a bacterial strain which comprises a 16S polynucleotide having 100% sequence identity to SEQ ID NO: 1 (see Table 2) to the plant, a part thereof, a seed for growing the plant, or a locus of the plant, wherein the microbial active ingredient is a supernatant from a culture of the bacterial strain or an extract or extract fraction from a culture of the bacterial strain.

A further aspect provides the use of bacteria of a bacterial strain which comprises a 16S polynucleotide having 100% sequence identity to SEQ ID NO: 1 or of a microbial active ingredient that is derived from said bacterial strain for conferring a pathogen control or resistance in a plant. In certain preferred embodiments, disease severity and/or the development of necrosis may be reduced or decreased by virtue of the present methods and uses.

The inventors also identified novel bacterial strains particularly advantageous in the present context. An aspect thus provides a bacterial strain as deposited under the Budapest Treaty at the Polish Collection of Microorganisms (PCM) on 11 Feb. 2022 under Accession No. B/00366, or a functional mutant thereof. For reasons of brevity, this strain (also see the particulars in Table 2) is also referred to as strain “MED-B-M14B4” (which corresponds to the identification reference given to the deposited material by the depositor) or strain “M14B4” throughout this specification. An aspect provides a bacterial strain as deposited under the Budapest Treaty at the Polish Collection of Microorganisms (PCM) on 26 Nov. 2021 under Accession No. B/00364, or a functional mutant thereof. For reasons of brevity, this strain (also see the particulars in Table 2) is also referred to as strain “MED-B-M2F4” (which corresponds to the identification reference given to the deposited material by the depositor) or strain “M2F4” throughout this specification. An aspect provides a bacterial strain as deposited under the Budapest Treaty at the at the Polish Collection of Microorganisms (PCM) on 2 Nov. 2022 under Accession No. B/00435, or a functional mutant thereof. For reasons of brevity, this strain (also see the particulars in Table 2) is also referred to as strain “MED-B-M14B2” (which corresponds to the identification reference given to the deposited material by the depositor) or strain “M14B2” throughout this specification. An aspect provides a bacterial strain as deposited under the Budapest Treaty at the Polish Collection of Microorganisms (PCM) on 2 Nov. 2022 under Accession No. B/00436, or a functional mutant thereof. For reasons of brevity, this strain (also see the particulars in Table 2) is also referred to as strain “MED-B-M14B3” (which corresponds to the identification reference given to the deposited material by the depositor) or strain “M14B3” throughout this specification. An aspect provides a bacterial strain as deposited under the Budapest Treaty at the Polish Collection of Microorganisms (PCM) on 2 Nov. 2022 under Accession No. B/00437, or a functional mutant thereof. For reasons of brevity, this strain (also see the particulars in Table 2) is also referred to as strain “MED-B-M14B1” (which corresponds to the identification reference given to the deposited material by the depositor) or strain “M14B1” throughout this specification. An aspect provides a bacterial strain as deposited under the Budapest Treaty at the Polish Collection of Microorganisms (PCM) on 2 Nov. 2022 under Accession No. B/00438, or a functional mutant thereof. For reasons of brevity, this strain (also see the particulars in Table 2) is also referred to as strain “MED-B-M2H3” (which corresponds to the identification reference given to the deposited material by the depositor) or strain “M2H3” throughout this specification. Also provided are combinations of two or more of these bacterial strains.

Further aspects provide: a bacterial population comprising one or more of the aforementioned strains; as well as an agricultural active composition comprising one or more of the aforementioned strains, or a microbial active ingredient capable of conferring a pathogen control or resistance in a plant wherein said ingredient is derived from the aforementioned bacterial strain.

Another aspect provides a plant or part thereof treated with said bacteria or with a composition comprising the bacteria; or a plant or part thereof heterologously disposed with said bacteria, or a plant or part thereof coated with said bacteria or with a composition comprising the bacteria or with the agricultural active composition as mentioned herein.

The bacterial strains, products, methods, and uses of the present invention advantageously allow to improve pathogen control and resistance in a plant, such as reducing the disease severity and/or necrosis caused by a pathogen in a plant. Hence, the herein described bacterial strains provide several significant advantages to plants, in particular to agricultural plants, such as wheat, barley, maize, cucurbits, and the like. For example, the disease severity and/or necrosis of a plant can be reduced compared to untreated plants by applying the teachings of the present invention. The present invention can thus allow to substitute or even abolish the use of chemical products such as chemical pesticides, including chemical fungicides or bactericides, and thereby advantageously facilitate more sustainable agriculture or increase the yield under adverse conditions. The teachings of the present invention can be immediately applied to any plant and, compared to provision of transgenic plants, do not require additional time for gene identification, generation and characterization of transgenic lines. Compared to the use of traditional agricultural methods including the application of chemical pesticides, the present approaches can require less resources, can be less labor intensive, and are more environmentally friendly, and thereby also compatible with organic farming practices.

The above and further aspects and preferred embodiments of the invention are described in the following sections and in the appended claims. The subject-matter of appended claims is hereby specifically incorporated in this specification.

### DESCRIPTION OF THE DRAWINGS

The following description of the figures of specific embodiments of the invention is merely exemplary in nature and is not intended to limit the present teachings, their application or uses.

FIG. 1 shows a graphical representation of the disease severity (%) of wheat plants upon inoculation with the fungal pathogen Fusarium graminearum (Fg) (disease control) compared to inoculation with Fg and an isolated bacterial strain according to embodiments of the present invention, and compared to a formulation control with no pathogen applied later (Mock). Bacterial strains applied here were B/00364, M14B1, M14B3, or B/00366. All p-values were <0.001.

FIG. 2 shows a graphical representation of the disease severity (%) of wheat plants upon inoculation with the fungal pathogen Puccinia striiformis var. tritici (Pst) (disease control) compared to inoculation with Pst and an isolated bacterial strain according to embodiments of the present invention, and compared to a formulation control with the pathogen also applied later (Formulation). Bacterial strains applied here were B/00364, M14B1, M14B2, M14B3, B/00366 or M2H3. All p-values were <0.001.

FIG. 3A-3C shows graphical representations of the disease severity (% of necrosis) of wheat plants upon inoculation with the fungal pathogen Zymoseptoria tritici (Disease control) compared to inoculation with Z. tritici and isolated bacterial strains according to embodiments of the present invention. Bacterial strains applied here were B/00364 (FIG. 3A), M14B2 (FIG. 3B), or B/00366 (FIG. 3C). All p-values were <0.001.

FIG. 4A shows a graphical representation of the reduction of the fungal disease fusarium head blight severity caused by Fusarium sp. in Triticum sp. on a field in Poland in 2022. The figure on the left side visualizes on the Y-axis the severity of the disease for untreated wheat plants and wheat plants treated with the bacterial strain M2F4. The figure on the right side shows the reduction of disease severity of wheat plants treated with the bacterial strain M2F4 in comparison to untreated wheat plants.

FIG. 4B shows a graphical representation of the reduction of the fungal disease fusarium head blight severity caused by Fusarium sp. in Triticum sp. on a field in Bulgaria in 2022. The figure on the left side visualizes on the Y-axis the severity of the disease for untreated wheat plants and wheat plants treated with the bacterial strain M2F4. The figure on the right side shows the reduction of disease severity of wheat plants treated with the bacterial strain M2F4 in comparison to untreated wheat plants.

FIG. 4C shows a graphical representation of the reduction of the fungal disease powdery mildew severity caused by Blumeria graminis f. sp. tritici in Triticum sp. on a field in France in 2022. The figure on the left side visualizes on the Y-axis the severity of the disease for untreated wheat plants and wheat plants treated with the bacterial strain M2F4. The figure on the right side shows the reduction of disease severity of wheat plants treated with the bacterial strain M2F4 in comparison to untreated wheat plants.

FIG. 4D shows a graphical representation of the reduction of the fungal disease powdery mildew severity caused by Blumeria graminis f. sp. tritici in Triticum sp. on a field in Poland in 2022. The figure on the left side visualizes on the Y-axis the severity of the disease for untreated wheat plants and wheat plants treated with the bacterial strain M2F4. The figure on the right side shows the reduction of disease severity of wheat plants treated with the bacterial strain M2F4 in comparison to untreated wheat plants.

FIG. 5 shows a graphical representation of the reduction of the fungal disease powdery mildew severity caused by Podosphaera xanthii in cucurbits (cucumber) on a field in Poland in 2022. The figure on the left side visualizes on the Y-axis the severity of the disease for untreated cucumber plants and cucumber plants treated with the bacterial strain M2F4. The figure on the right side shows the reduction of disease severity of cucumber plants treated with the bacterial strain M2F4 in comparison to untreated cucumber plants.

### DETAILED DESCRIPTION OF THE INVENTION

As used herein, the singular forms “a”, “an”, and “the” include both singular and plural referents unless the context clearly dictates otherwise.

The terms “comprise”, “comprising”, “comprises” and “comprised of” as used herein are synonymous with “include”, “including”, “includes” or “contain”, “containing”, “contains”, and are inclusive or open-ended and do not exclude additional, non-recited members, elements or method steps. The terms also encompass “consisting of” and “consisting essentially of”.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

Whereas the term “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims. All documents cited in the present specification are hereby incorporated by reference in their entirety.

Throughout this disclosure, various publications, patents and published patent specifications may be referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

By extensive experimental testing, the present inventors have found that certain bacterial strains exhibit pathogen control or resistance effects in plants and hence that such strains can advantageously be used as pathogen control products on plants. In particular the bacterial strains provided an unexpected decrease in plant disease features such as disease severity and necrosis caused by pathogens in the tested plants as compared to untreated plants.

Accordingly, an aspect of the invention relates to a method for conferring a pathogen control or resistance in a plant, the method comprising administering bacteria of a bacterial strain which comprises a 16S polynucleotide having 100% sequence identity to SEQ ID NO: 1 to the plant, a part thereof, a seed for growing the plant, or a locus of the plant. For example, such methods may be suitably practiced in the context of agriculture or horticulture.

Also provided is a method for conferring a pathogen control or resistance in a plant, the method comprising administering a microbial active ingredient that is derived from a bacterial strain which comprises a 16S polynucleotide having 100% sequence identity to SEQ ID NO: 1 to the plant, a part thereof, a seed for growing the plant, or a locus of the plant, wherein the microbial active ingredient is a supernatant from a culture of the bacterial strain or an extract or extract fraction from a culture of the bacterial strain. Such methods may, for example, be suitably practiced in the context of agriculture or horticulture.

A plant or part thereof treated with said bacteria or with a composition comprising the bacteria; or a plant or part thereof heterologously disposed with said bacteria, or a plant or part thereof coated with a composition comprising the bacteria, are also provided herein.

A further aspect discloses a bacterial strain selected from the group consisting of a bacterial strain as deposited under the Budapest Treaty at the at the Polish Collection of Microorganisms (PCM) on 11 Feb. 2022 under Accession No. B/00366 (with the following identification reference given to the deposited material by the depositor “MED-B-M14B4”), or a functional mutant thereof, a bacterial strain as deposited under the Budapest Treaty at the at the Polish Collection of Microorganisms (PCM) on 21 Nov. 2021 under Accession No. B/00364 (with the following identification reference given to the deposited material by the depositor “MED-B-M2F4”), or a functional mutant thereof, or a bacterial strain as deposited under the Budapest Treaty at the at the Polish Collection of Microorganisms (PCM) on 2 November under Accession No. B/00435 (with the following identification reference given to the deposited material by the depositor “MED-B-M14B2”), or a functional mutant thereof, or a bacterial strain as deposited under the Budapest Treaty at the at the Polish Collection of Microorganisms (PCM) on 2 November under Accession No. B/00436 (with the following identification reference given to the deposited material by the depositor “MED-B-M14B3”), or a functional mutant thereof, or a bacterial strain as deposited under the Budapest Treaty at the at the Polish Collection of Microorganisms (PCM) on 2 November under Accession No. B/00437 (with the following identification reference given to the deposited material by the depositor “MED-B-M14B1”), or a functional mutant thereof, or a bacterial strain as deposited under the Budapest Treaty at the at the Polish Collection of Microorganisms (PCM) on 2 November under Accession No. B/00438 (with the following identification reference given to the deposited material by the depositor “MED-B-M2H3”), or a functional mutant thereof.

Another aspect discloses a bacterial strain selected from the group consisting of a bacterial strain as deposited under the Budapest Treaty at the at the Polish Collection of Microorganisms (PCM) on 11 Feb. 2022 under Accession No. B/00366 (with the following identification reference given to the deposited material by the depositor “MED-B-M14B4”), a bacterial strain as deposited under the Budapest Treaty at the at the Polish Collection of Microorganisms (PCM) on 21 Nov. 2021 under Accession No. B/00364 (with the following identification reference given to the deposited material by the depositor “MED-B-M2F4”), or a bacterial strain as deposited under the Budapest Treaty at the at the Polish Collection of Microorganisms (PCM) on 2 November under Accession No. B/00435 (with the following identification reference given to the deposited material by the depositor “MED-B-M14B2”), or a bacterial strain as deposited under the Budapest Treaty at the at the Polish Collection of Microorganisms (PCM) on 2 November under Accession No. B/00436 (with the following identification reference given to the deposited material by the depositor “MED-B-M14B3”), or a bacterial strain as deposited under the Budapest Treaty at the at the Polish Collection of Microorganisms (PCM) on 2 November under Accession No. B/00437 (with the following identification reference given to the deposited material by the depositor “MED-B-M14B1”), or a bacterial strain as deposited under the Budapest Treaty at the at the Polish Collection of Microorganisms (PCM) on 2 November under Accession No. B/00438 (with the following identification reference given to the deposited material by the depositor “MED-B-M2H3”). The proposed taxonomic designation of this strain is Streptomyces bottropensis.

Also provided are combinations comprising any of these bacterial strains. Further provided is a bacterial population comprising one or more of the aforementioned strains, as well as an agricultural active composition comprising one or more of the aforementioned strains. As used herein, the term “bacterium”, “bacteria”, or “bacterial” refers in general to any prokaryotic organism, and may refer to an organism from either Kingdom Eubacteria (Bacteria), Kingdom Archaebacteria (Archaea), or both. In some cases, bacterial genera have been reassigned due to various reasons (such as, but not limited to, the evolving field of whole genome sequencing), and it is understood that such nomenclature reassignments are within the scope of any claimed genus.

As used herein, “bacterial strain” (which may be abridged to “strain” where the context makes clear that a bacterial strain is meant) refers to any of the prokaryotic microorganism belonging to the same class of species, including the species. The term “strain” as a basic operational unit of microbial taxonomy, such as bacterial taxonomy, is frequently used to denote a population made up of the descendants of a single isolation in pure culture, usually made up of a succession of cultures ultimately derived from an initial single colony. Where a species encompasses two or more distinct isolates, the term “strain” may be used to refer to an isolate or group of isolates that can be distinguished from other isolates of the same genus and species by phenotypic characteristics or genotypic characteristics or both.

In the practice of the present invention, the strain may be deemed as “isolated” or “purified”. The terms “isolated” or “purified” with reference to a particular component generally denote that such component exists in separation from—for example, has been separated from or prepared and/or maintained in separation from—one or more other components of its natural environment. The terms do not necessarily reflect the extent to which the component has been purified. Hence, the phrases “isolated bacterial strain” or “purified bacterial strain” may be seen as referring to a strain that has been removed from its natural milieu. In particular, the terms refer to substantially no other strains than the desired strain, which is thus substantially free of other contaminants, which can include microbial contaminants. Further, the terms may denote that the strain has been separated from materials with which it is normally found in nature. A strain heterologously disposed to other strains, or with compounds or materials with which it is not normally found in nature, is encompassed by the phrases “isolated bacterial strain” or “purified bacterial strain”.

In certain embodiments, the purified bacterial strains as taught herein may become or behave as endophytes. An “endophyte” is an organism capable of living on a plant element (e.g., rhizoplane or phyllosphere) or within a plant element (e.g., endosphere) or on a surface in close physical proximity with a plant element (e.g., the rhizosphere or on a seed). Endophytes can occupy the intracellular or extracellular spaces of plant tissue, including but not limited to leaves, stems, flowers, fruits, seeds, or roots. An endophyte can be, for example, a bacterial or fungal organism, and can confer a beneficial property to the host plant such as an increase in yield, biomass, resistance, and/or fitness. An endophyte can be a bacterium or a fungus. As used herein, the term “microbe” or “strain” is sometimes used to describe an endophyte. As used herein, the microbes or strains as described herein can be labelled as endophytes.

Endophytes may favorably impact one or more traits of agronomic interest in plants. By means of an example and without limitation, a plant heterologously disposed with one or more endophyte microorganism, or a plant grown from a plant part or seed treated with or heterologously disposed with one or more endophyte microorganism, such as an endophytic bacterial or fungal strain, may exhibit a trait of agronomic interest, such as a trait selected from the group consisting of: disease resistance, reduced necrosis, drought tolerance, heat tolerance, cold tolerance, salinity tolerance, metal tolerance, herbicide tolerance, chemical tolerance, improved water use efficiency, improved phosphorus solubilization, improved phosphorus mobilization, improved nitrogen utilization, improved nitrogen fixation, pest resistance, herbivore resistance, pathogen resistance, increase in yield, increase in yield under water-limited conditions, health enhancement, vigor improvement, growth improvement, improved plant emergence, photosynthetic capability improvement, nutrition enhancement, altered protein content, altered oil content, increase in biomass, increase in number of tillers per plant, increase in shoot length, increase in root length, improved root architecture, increase in seed weight, altered seed carbohydrate composition, altered seed oil composition, increase in radical length, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, reduced number of wilted leaves per plant, reduced number of severely wilted leaves per plant, increase in number of non-wilted leaves per plant, improved plant visual appearance, and combinations thereof.

As used herein, a microorganism, such as a bacterial or fungal strain, such as an endophytic bacterial or fungal strain, is considered to have conferred pathogen control or resistance whether or not the improvement arose from the plant, the strain, or the concerted action between the plant and the strain. Therefore, for example, where pathogen control or resistance results at least in part from the production of a beneficial hormone or chemical, for the purposes of the present specification the strain will be considered to have conferred pathogen control or resistance upon the plant as compared to a plant, plant part or seed that has not been treated with or heterologously disposed with said strain, whether the beneficial hormone or chemical is produced by the plant or by the strain. In certain embodiments, the pathogen control or resistance may be directly attributable to the microbial strain, rather than to the effect of the strain on the biochemistry of the plant.

Particularly envisaged herein is the administration of live bacteria and/or microorganisms. The term “live” as used herein is synonymous with “viable” and refers to any living intact state of a microorganism, such as active growth or dormancy, from which state it can multiply and/or reproduce itself in a medium capable of supporting the growth of the microorganism. Typically, substantially all bacteria or microorganisms comprised by populations or compositions intended herein may be live or viable. For example, at least 50%, preferably at least 60%, more preferably at least 75%, still more preferably at least 90%, such as at least 95%, 96%, 97%, 98%, 99% or 100% of the bacteria or microorganisms in the population or composition may be viable, such as capable of forming colonies when plated on a suitable solid medium.

The term “16S polynucleotide”, or synonymous terms such as “16S nucleotide sequence” or “16S”, refer to the nucleic acid sequence, such as in particular the DNA sequence, of the 16S ribosomal RNA (rRNA) of a bacterium. 16S rRNA gene sequencing is a well-established method for studying phylogeny and taxonomy of bacteria. A full length 16S nucleic acid sequence is approximately 1500 nucleotides in length. In certain embodiments, the bacterial strain may comprise a single copy of the 16S rRNA gene. In certain embodiments, the bacterial strain may comprise more than one copy of the 16S rRNA gene, such as two, three or more (multicopy) copies of the 16S rRNA gene. In such embodiments where two or more 16S rRNA gene copies are found in the bacterial strain, at least one of these 16S rRNA gene copies complies with the sequence identity requirements specified in the present application, preferably two or more and preferably all of the 16S rRNA gene copies (each independently) comply with the stated sequence identity requirements. By means of an example and without limitation, where a bacterial strain comprises three 16S rRNA gene copies, at least one, preferably at least two, and more preferably all three 16S rRNA gene copies will, each independently, display 100% sequence identity to SEQ ID NO: 1. Conveniently, the 16S rRNA sequence can be determined by sequencing (e.g., Sanger sequencing) the 16S gene sequence(s) in the chromosomal DNA, which may be amplified (e.g., PCR amplified) using suitable amplification primers, such as in particular the Forward primer 90F: AAACT[C/T]AAA[T/G]GAATTGACGG (SEQ ID NO: 2) and Reverse primer 1100R: GTTGCTCGCGTTGGGA (SEQ ID NO: 3).

The terms “identity”, “sequence identity” or “identical” in the context of nucleotide sequences may be used interchangeably herein, and refer to the extent that nucleic acid sequences are identical on a nucleotide-by-nucleotide basis, over a window of comparison. The percentage of sequence identity may be calculated by comparing two optimally aligned sequences over the window of comparison, determining the number of positions at which the identical nucleic acid base occurs in both sequences to yield the number of matched positions, dividing the number of matched positions by the total number of positions in the window of comparison, and multiplying the result by 100 to yield the percentage of sequence identity. The percent identity value may, but need not, be rounded to the nearest tenth. For example, 98.11, 98.12, 98.13, and 98.14 may be rounded down to 98.1, while 98.15, 98.16, 98.17, 98.18, and 98.19 may be rounded up to 98.2.

Sequence identity between nucleic acids as envisaged herein may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215:403-10), such as the “Blast 2 sequences” tool described by Tatusova and Madden 1999 (FEMS Microbiol Lett 174:247-250), or the “blastn suite-2sequences” sequence alignment algorithm described by Zheng Zhang et al. 2000 (J Comput Biol 2000, vol. 7 (1-2), 203-14), now incorporated into the BLAST program suite available at ncbi.nlm.nih.gov. The skilled person can implement such algorithms and set the requisite parameters. By means of an example and without limitation, parameters for the BLASTN program may be as follows: cost to open a gap=0, cost to extend a gap=2.5, reward for a match=1, penalty for a mismatch=−2, Expect value=0.05, word size=28, Low Complexity Filter=Yes.

There are further algorithms known in the art that can be used to measure nucleotide sequence identity. Nucleotide sequence identity can be measured by a local or global alignment, preferably implementing an optimal local or optimal global alignment algorithm. For example, a global alignment may be generated using an implementation of the Needleman-Wunsch algorithm (Needleman & Wunsch. Journal of Molecular Biology 1970, vol. 48 (3), 443-53). For example, a local alignment (which does not consider the entirety of the sequence but tries to find the longest subsequence that confirms to a given matching criteria) may be generated using an implementation of the Smith-Waterman algorithm (Smith & Waterman Journal of Molecular Biology 1981, vol. 147 (1), 195-197). Optimal global alignments using the Needleman-Wunsch algorithm and optimal local alignments using the Smith-Waterman algorithm are implemented in USEARCH (https://www.drive5.com/usearch/), for example USEARCH version 11.0.667.

A gap is a region of an alignment wherein a sequence does not align to a position in the other sequence of the alignment. In global alignments, terminal gaps are discarded before identity is calculated. For both local and global alignments, internal gaps are counted as differences. A terminal gap is a region beginning at the end of a sequence in an alignment wherein the nucleotide in the terminal position of that sequence does not correspond to a nucleotide position in the other sequence of the alignment and extending for all contiguous positions in that sequence wherein the nucleotides of that sequence do not correspond to a nucleotide position in the other sequence of the alignment.

Sequence identity as envisaged herein in particular denotes overall sequence identity, i.e., sequence identity calculated from optimally aligning the whole sequences of the to-be-compared 16S rRNA genes. In other words, the nucleic acid sequences to be aligned are the complete 16S rRNA genes, and the window of comparison corresponds to the whole region of optimal alignment between these complete 16S sequences, i.e., to the alignment length. Hence, in an example, a query 16S rRNA gene sequence, such as the sequence set forth in SEQ ID NO: 1, is optimally aligned with another 16S rRNA gene sequence (in case of a global alignment any terminal gaps are disregarded; in case of a local alignment the region of alignment will be expressed as the region between a given 5′ and a given 3′ position in the query sequence), and the percentage sequence identity is calculated over a window of comparison which corresponds to the whole region of alignment, counting internal gaps as differences.

Bacterial strains as envisaged herein preferably comprise a 16S polynucleotide the length of which is between 90% and 110% (1371-1675 nucleotides), more preferably between 95% and 105% (1447-1599 nucleotides) of the length of the 16S region polynucleotide shown in SEQ ID NO: 1. These 16S sequences can be subjected to pairwise sequence comparisons with SEQ ID NO: 1.

When two complete 16S region sequences in a pairwise sequence comparison are optimally aligned, for example by a local alignment algorithm such as BLAST, it is particularly envisaged that the alignment length is at least 90% of the length of the shorter one of the two 16S sequences, preferably at least about 91%, 92%, 93%, 94%, more preferably at least about 95%, or at least about 96%, 97%, 98%, 99% or 100% of the length of the shorter one of the two 16S sequences, and the window of comparison corresponds to the whole alignment length.

Preferably, the region of alignment, for example the region of alignment provided by a local alignment algorithm such as BLAST, will comprise at least 90% of the length of SEQ ID NO: 1, more preferably at least about 91%, 92%, 93%, 94%, even more preferably at least about 95%, or at least about 96%, 97%, 98%, 99% or 100% of the length of SEQ ID NO: 1. Preferably, the region of alignment, for example the region of alignment provided by a local alignment algorithm such as BLAST, will comprise at least 1371 contiguous (the term contiguous in this context does not exclude the presence of internal gaps in the alignment) nucleotides of SEQ ID NO: 1, more preferably at least 1380 contiguous nucleotides, or at least 1390 contiguous nucleotides, or at least 1400 contiguous nucleotides, such as at least 1410, at least 1420, at least 1430, or at least 1440 contiguous nucleotides of SEQ ID NO: 1. Particularly preferably, the region of alignment will comprises at least 1447, or in increasing order of preference, at least 1450, at least 1460, at least 1470, at least 1480, at least 1490, at least 1500, at least 1510, at least 1520, or all 1523 contiguous nucleotides of SEQ ID NO: 1.

In certain preferred embodiments, the bacterial strain comprises a 16S polynucleotide having at 100% sequence identity to SEQ ID NO: 1.

In certain particularly preferred embodiments, the bacterial strain comprises a 16S polynucleotide which, when aligned over a region of alignment comprising at least 1447 contiguous nucleotides of SEQ ID NO: 1, or in increasing order of preference, over a region of alignment comprising at least 1450, at least 1460, at least 1470, at least 1480, at least 1490, at least 1500, at least 1510, at least 1520, or all 1523 contiguous nucleotides of SEQ ID NO: 1, will display no nucleotide mismatches and internal gaps with SEQ ID NO: 1. The mismatch or internal gap in this context refers to a single nucleotide mismatch or a gap that involves or spans a single nucleotide.

In certain particularly preferred embodiments, the bacterial strain comprises a 16S polynucleotide as set forth in (i.e., identical to) SEQ ID NO: 1, i.e., a 16S polynucleotide identical over the full length thereof to SEQ ID NO: 1.

In certain embodiments, the bacterial strain is a Streptomyces bottropensis strain. Streptomyces bottropensis is in particular known to include aerobic, Gram-positive, non-motile, chemo-organotrophic actinomycetes.

In certain embodiments, the bacterial strain as envisaged herein is the strain as deposited under the Budapest Treaty at the at the Polish Collection of Microorganisms (PCM) on 11 Feb. 2022 under Accession No. B/00366, or a functional mutant thereof. For reasons of brevity, this strain (also see the particulars in Table 2) is also referred to as strain “MED-B-M14B4” (which corresponds to the identification reference given to the deposited material by the depositor) or strain “M14B4” throughout this specification. In certain embodiments, the bacterial strain as envisaged herein is the strain as deposited under the Budapest Treaty at the at the Polish Collection of Microorganisms (PCM) on 11 Feb. 2022 under Accession No. B/00366.

In certain embodiments, the bacterial strain as envisaged herein is the strain as deposited under the Budapest Treaty at the at the Polish Collection of Microorganisms (PCM) on 26 Nov. 2021 under Accession No. B/00364, or a functional mutant thereof. For reasons of brevity, this strain (also see the particulars in Table 2) is also referred to as strain “MED-B-M2F4” (which corresponds to the identification reference given to the deposited material by the depositor) or strain “M2F4” throughout this specification. In certain embodiments, the bacterial strain as envisaged herein is the strain as deposited under the Budapest Treaty at the at the Polish Collection of Microorganisms (PCM) on 26 Nov. 2021 under Accession No. B/00364.

In certain embodiments, the bacterial strain as envisaged herein is the strain as deposited under the Budapest Treaty at the at the Polish Collection of Microorganisms (PCM) on 2 Nov. 2022 under Accession No. B/00435, or a functional mutant thereof. For reasons of brevity, this strain (also see the particulars in Table 2) is also referred to as strain “MED-B-M14B2” (which corresponds to the identification reference given to the deposited material by the depositor) or strain “M14B2” throughout this specification. In certain embodiments, the bacterial strain as envisaged herein is the strain as deposited under the Budapest Treaty at the at the Polish Collection of Microorganisms (PCM) on 2 Nov. 2022 under Accession No. B/00435.

In certain embodiments, the bacterial strain as envisaged herein is the strain as deposited under the Budapest Treaty at the at the Polish Collection of Microorganisms (PCM) on 2 Nov. 2022 under Accession No. B/00436, or a functional mutant thereof. For reasons of brevity, this strain (also see the particulars in Table 2) is also referred to as strain “MED-B-M14B3” (which corresponds to the identification reference given to the deposited material by the depositor) or strain “M14B3” throughout this specification. In certain embodiments, the bacterial strain as envisaged herein is the strain as deposited under the Budapest Treaty at the at the Polish Collection of Microorganisms (PCM) on 2 Nov. 2022 under Accession No. B/00436.

In certain embodiments, the bacterial strain as envisaged herein is the strain as deposited under the Budapest Treaty at the at the Polish Collection of Microorganisms (PCM) on 2 Nov. 2022 under Accession No. B/00437, or a functional mutant thereof. For reasons of brevity, this strain (also see the particulars in Table 2) is also referred to as strain “MED-B-M14B1” (which corresponds to the identification reference given to the deposited material by the depositor) or strain “M14B1” throughout this specification. In certain embodiments, the bacterial strain as envisaged herein is the strain as deposited under the Budapest Treaty at the at the Polish Collection of Microorganisms (PCM) on 2 Nov. 2022 under Accession No. B/00437.

In certain embodiments, the bacterial strain as envisaged herein is the strain as deposited under the Budapest Treaty at the at the Polish Collection of Microorganisms (PCM) on 2 Nov. 2022 under Accession No. B/00438, or a functional mutant thereof. For reasons of brevity, this strain (also see the particulars in Table 2) is also referred to as strain “MED-B-M2H3” (which corresponds to the identification reference given to the deposited material by the depositor) or strain “M2H3” throughout this specification. Also provided are combinations of two or more of these bacterial strains. In certain embodiments, the bacterial strain as envisaged herein is the strain as deposited under the Budapest Treaty at the at the Polish Collection of Microorganisms (PCM) on 2 Nov. 2022 under Accession No. B/00438.

In certain embodiments, a combination of one or more of the aforementioned bacterial strains is envisaged herein.

In preferred embodiments, the bacterial strain as envisaged herein is the strain as deposited under the Budapest Treaty at the at the Polish Collection of Microorganisms (PCM) on 11 Feb. 2022 under Accession No. B/00366, or a functional mutant thereof.

The term “functional mutant” means a bacterial strain directly or indirectly obtained by genetic modification (such as by random mutagenesis or by targeted genetic modification) of the respective referenced strain and retaining at least some extent of the activity of the referenced strain on the plant pathogen control or resistance, preferably retaining at least 10%, such as at least 20%, at least 30%, or at least 40%, preferably at least 50%, such as at least 60%, at least 70%, or at least 80%, more preferably at least 90%, such as 100%, or even greater than 100% of the activity of the referenced strain on the plant pathogen control or resistance. The genetic modification of a functional mutant can be achieved through any means, such as, but not limited to, chemical mutagens, ionizing radiation, transposon-based mutagenesis, or via conjugation, transduction, or transformation using the referenced strains as either the recipient or donor of genetic material. In certain embodiments, the 16S rRNA gene sequence of the functional mutant remains identical to the 16S rRNA gene sequence of the referenced strain. Hence, the functional mutant may preferably comprise the 16S rRNA gene sequence as shown in SEQ ID NO: 1. In certain embodiments, the functional mutant comprises at most 10, such as in increasing order of preference, at most 9, 8, 7, 6, 5, 4, 3, 2, or at most 1 chromosomal loci (such as genes) whose nucleic acid sequence differs from (has been modified compared to) the sequence of the corresponding loci in the referenced strain, and/or the functional mutant comprises at most 10, such as in increasing order of preference, at most 9, 8, 7, 6, 5, 4, 3, 2, or at most 1 transgenic elements (such as transgenes) introduced into it and not present in the referenced strain. In certain embodiments, at most 10, such as in increasing order of preference, at most 9, 8, 7, 6, 5, 4, 3, 2, or at most 1 gene of the functional mutant carries a non-synonymous mutation not present in the reference strain. Hence, also disclosed is a method of genetically modifying the bacterial strain as envisaged herein, such as by mutagenesis, for example random or directed mutagenesis, and/or by transgenesis, for example by the introduction of one or more transgenes thereto, whereby a functional mutant of the referenced strain is obtained.

In certain embodiments, bacterial strains as taught herein may be provided with a reporter gene for purposes of tracking the microorganisms. Examples of reporter genes encode luciferase, (green/red) fluorescent protein and variants thereof, like EGFP (enhanced green fluorescent protein), RFP (red fluorescent protein, like DsRed or DsRed2), CFP (cyan fluorescent protein), BFP (blue green fluorescent protein), YFP (yellow fluorescent protein), β-galactosidase or chloramphenicol acetyltransferase, and the like. Preferably the reporter gene is a fluorescent gene, such as GFP. For example, GFP can be from Aequorea victoria. Other mutated forms of GFP include, but are not limited to, pRSGFP, EGFP, RFP/DsRed, and EYFP, BFP, YFP, among others, are commercially available. Such bacterial strains can be readily detected. Such bacterial strains can be readily quantified.

In certain embodiments, bacteria of two or more bacterial strains as described in the present specification may be administered to the plant, the part thereof, the seed for growing the plant, or the locus of the plant.

In certain embodiments, the bacteria as described in the present specification may be administered to the plant, the part thereof, the seed for growing the plant, or the locus of the plant in conjunction with one or more additional plant-beneficial microorganism. As used throughout the present specification, the term “microorganism” or “microbe” refers to any strain, any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism is a bacterial strain. In some embodiments, a microbe or microorganism is a fungal strain. In some embodiments, a microbe or microorganism is an endophyte, for example a bacterial or fungal endophyte, which is capable of living within a plant.

In some embodiments, a microbe or microorganism encompasses individual cells (e.g., unicellular microorganisms) or more than one cell (e.g., multi-cellular microorganism).

Diverse plant-associated microorganisms can positively impact plant health and physiology in a variety of ways. In particular, plant-beneficial microorganisms, when administered to a plant, plant part, a seed for growing a plant, or a locus of a plant may improve one or more traits of agronomic importance in a plant, such as one or more plant growth features, or may improve the plant's pathogen resistance. Where the improved trait can be quantified, any extent of an improvement is contemplated. For example, a plant-beneficial microorganism may provide an improved trait of agronomic importance in a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with a reference plant grown under the same conditions. By means of an illustration, a plant-beneficial microorganism may be capable of increasing nutrient uptake and/or nutrient use efficiency of a treated plant as compared to an untreated plant, increasing the nitrogen fixating capacities or phosphorus uptake of a treated plant as compared to an untreated plant, increasing the amount of biomass of a treated plant as compared to an untreated plant, increasing the number of tillers per plant of a treated plant as compared to an untreated plant, increasing growth and/or yield of a treated plant as compared to an untreated plant, and/or helping a treated plant overcome stress conditions, such as nutrient stress, compared to an untreated plant; and the like. Further traits of agronomic importance that can be improved by plant-beneficial microorganisms may include disease resistance, drought tolerance, heat tolerance, cold tolerance, salinity tolerance, metal tolerance, herbicide tolerance, chemical tolerance, improved water use efficiency, improved phosphorus solubilization, improved phosphorus mobilization, improved nitrogen utilization, improved nitrogen fixation, pest resistance, herbivore resistance, pathogen resistance, increase in yield, increase in yield under water-limited conditions, health enhancement, vigor improvement, growth improvement, improved plant emergence, photosynthetic capability improvement, nutrition enhancement, altered protein content, altered oil content, increase in biomass, increase in number of tillers per plant, increase in shoot length, increase in root length, improved root architecture, increase in seed weight, altered seed carbohydrate composition, altered seed oil composition, increase in radical length, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, reduced number of wilted leaves per plant, reduced number of severely wilted leaves per plant, increase in number of non-wilted leaves per plant, and/or improved plant visual appearance, and the like.

By means of an illustration and without limitation, plant-beneficial microorganisms may include mycorrhizal fungi, including endomycorrhizal fungi and ectomycorrhizal fungi, such as fungi belonging to the divisions Basidiomycota, Ascomycota, and Zygomycota, bacteria of the family Rhizobiaceae, bacteria of the genera Frankia, Azotobacter, Azospirillum, Acetobacter, Azoarcus, Burkholderia, Herbaspirillum, Pseudomonas (e.g., Pseudomonas fluorescens, P. putida, P. gladioli), Bacillus (Bacillus subtilis, B. cereus, B. circulans), further bacteria such as Serratia marcescens, Flavobacterium spp., Alcaligenes sp., Agrobacterium radiobacter, Streptomyces tendae, Streptomyces marokkonesis, Stenotrophomonas rhizophila, and others. In certain embodiments, the plant-beneficial microorganisms are selected from those disclosed in WO2018060519, WO2020161351, WO2020161352, WO2023170281 and WO2023126460.

In certain embodiments, the one or more additional plant-beneficial microorganism may be selected to improve the efficacy of the bacterial strain as taught herein, in particular efficacy in improving the plant pathogen control or resistance conferred by the bacterial strain. Hence, also provided is a method of improving the efficacy of the bacterial strain as taught herein, comprising the selection of an additional plant-beneficial microorganism, whereby co-administration of the additional plant-beneficial microorganism with the bacterial strain or strains to a plant, plant part, or seed improves the plant pathogen control or resistance conferred by the bacterial strain. In such embodiments, the administration of the plant-beneficial microorganism alone may but need not lead to an improvement in a plant trait.

Without wishing to be limitative, the present bacteria can be administered in conjunction with a fungal inoculant, said fungal inoculant can comprise a fungal inoculant of the family Glomeraceae, a fungal inoculant of the family Claroidoglomeraceae, a fungal inoculant of the family Acaulosporaceae, a fungal inoculant of the family Sacculospraceae, a fungal inoculant of the family Entrophosporaceae, a fungal inoculant of the family Pacidsproraceae, a fungal inoculant of the family Diversisporaceae, a fungal inoculant of the family Paraglomeraceae, a fungal inoculant of the family Archaeosporaceae, a fungal inoculant of the family Geosiphonaceae, a fungal inoculant of the family Ambisporacea, a fungal inoculant of the family Scutellosproaceae, a fungal inoculant of the family Dentiscultataceae, a fungal inoculant of the family Racocetraceae, a fungal inoculant of the phylum Basidiomycota, a fungal inoculant of the phylum Ascomycota, a fungal inoculant of the phylum Zygomycota, a fungal inoculant of the genus Glomus, or a combination thereof.

Without wishing to be limitative, the present bacteria can be administered in conjunction with a bacterial inoculant, said bacterial inoculant can comprise a bacterial inoculant of the genus Rhizobium, bacterial inoculant of the genus Bradyrhizobium, bacterial inoculant of the genus Mesorhizobium, bacterial inoculant of the genus Azorhizobium, bacterial inoculant of the genus Allorhizobium, bacterial inoculant of the genus Burkholderia, bacterial inoculant of the genus Sinorhizobium, bacterial inoculant of the genus Kluyvera, bacterial inoculant of the genus Azotobacter, bacterial inoculant of the genus Pseudomonas, bacterial inoculant of the genus Azosprillium, bacterial inoculant of the genus Bacillus, bacterial inoculant of the genus Streptomyces, bacterial inoculant of the genus Paenibacillus, bacterial inoculant of the genus Paracoccus, bacterial inoculant of the genus Enterobacter, bacterial inoculant of the genus Alcaligenes, bacterial inoculant of the genus Mycobacterium, bacterial inoculant of the genus Trichoderma, bacterial inoculant of the genus Gliocladium, bacterial inoculant of the genus Klebsiella, or a combination thereof.

In certain embodiments, a microbial active ingredient that is derived from a bacterial strain as disclosed herein is presented. In some embodiments a microbial active ingredient is a supernatant from the culture wherein the bacterial strain has been cultured. The term “supernatant” refers to the liquid broth remaining when cells grown in said broth are removed by centrifugation, filtration, sedimentation or other means well known in the art. In some other embodiments, a microbial active ingredient is an extract or extract fraction from the culture wherein the bacterial strain has been cultured. The term “extract” refers to various forms of microbial products. Said microbial products are obtained by removing the cell walls and/or cell membranes of the bacterial cells, a process known as lysis, thereby obtaining one or more endogenous products of the cultured bacterial cells. In some embodiments, a microbial active ingredient is one or more endogenous products of the bacterial strain. Non-limiting examples of endogenous products are amino acids, peptides, enzymes, secondary metabolites, vitamins, minerals. In some embodiments, a metabolite produced by the bacterial strain is contemplated. In some embodiments, the microbial active ingredient is a cell-free or inactivated preparation of the bacterial strain as disclosed herein.

Removing the cell walls and/or cell membranes of the bacterial strain in culture can be performed by several procedures which are well-known by the person skilled in the art. Non-limiting examples are the addition of chemicals to the culture, heating the culture, or induce cell lysis in a mechanical way. An extract can also be obtained by autolysis of the bacterial strain.

In some embodiments, the composition comprises a pure bacterial population as active ingredient.

In some embodiments, the composition comprises one or more bacterial strains as disclosed herein as a mixture.

In certain embodiments, the bacteria can be comprised in or be part of an agricultural active composition. Hence, the methods may entail administering or applying such an agricultural active composition to the plant, the part thereof (e.g., roots), the seed for growing the plant, or the locus of the plant (e.g., to soil or plant growth medium surrounding the plant).

The term “composition” generally refers to a thing composed of two or more components, and more specifically denotes a combination or mixture of two or more materials, such as elements, molecules, substances, and/or microorganisms, as well as reaction products and decomposition products formed from the materials of the composition. The term may be interchangeably used with the terms “formulation” or “preparation”.

Agricultural active compositions typically comprise one or more agriculturally active ingredients and one or more agriculturally acceptable carrier or auxiliary. The terms “active ingredient” or “active component” can be used interchangeably and broadly refer to a material, such as an element, molecule, substance, and/or microorganism, which, when provided in an effective amount, achieves a desired outcome, such as achieves one or more effects on one or more traits of agronomic importance in plants. Typically, an active ingredient as intended herein may achieve such outcome(s) through interacting with and/or modulating the plant, part thereof, a seed for growing the plant, or the locus of the plant. The terms “agriculturally acceptable” or “agriculturally compatible” are consistent with the art and mean not deleterious to the recipient plant, such as not producing, having or causing any adverse effects when applied to a plant or to an organ, part or element of the plant, or adverse effects to the plant grown from that plant organ, part or element. The agriculturally active formulations may comprise materials which facilitate or enhance the stability, viability, storage, and/or administration of the bacterial strain(s) and/or plant-beneficial microorganism(s) as disclosed herein, and/or the colonization of the plant thereby.

Compositions as typically used herein may be liquid, semi-solid, or solid, and may include solutions or dispersions. Non-limiting examples of the compositions as taught herein may be soluble powders, soluble granules, wettable granules, tablet formulations, dry flowables, aqueous flowables, wettable dispersible granules, oil dispersions, suspension concentrates, dispersible concentrates, emulsifiable concentrates, aqueous suspensions, fertilizer granules, sprayables, and the like. In certain embodiments, a composition may be composed of components that are provided to an end user as a mixture, i.e., the composition components are already admixed. In certain embodiments, a composition may be composed of components one or more of which are provided to an end user in a physically separated form (e.g., in separate containers or vials) from one or more other components of the composition, although typically as part of the same product package or dispensing device. By means of an example and without limitation, the composition may comprise one or more components provided in one container, and one or more components provided in another container. Such arrangement allows the end user to admix the components of the composition shortly before use. For example, the composition may comprise the bacteria and optionally further plant beneficial microorganism(s) as taught herein provided in one container, and one or more auxiliaries provided in another container, to be admixed by the end user before use.

In certain embodiments, the composition comprises one or more agriculturally acceptable auxiliary. The terms “auxiliary”, “auxiliary agent”, “additive”, or “adjuvant” may be used interchangeably herein. The auxiliaries may be natural or synthetic organic or inorganic materials which facilitate the administration of actives to plants, plant parts, seeds, or plant growth loci. In certain embodiments, the auxiliaries may be one or more of as a solvent, a carrier, a surfactant, a sticker, an antifreeze agent, a thickener, a buffering agent, an antifoaming agent, an antioxidant, a preservative, an aroma, or a colorant. Suitable auxiliary agents and inert agents are known in the art and are commercially available. In general, the bacteria and optionally further plant-beneficial microorganism(s) can be combined with any solid, semi-solid or liquid additive customarily used for formulation purposes. A carrier is to be understood as meaning a natural or synthetic, organic or inorganic substance which is mixed or combined with the bacteria and optionally further plant-beneficial microorganism(s) for better applicability, in particular for application to plants or plant parts such as seeds. The carrier, which may be solid, semi-solid, or liquid, is generally inert and suitable for use in agriculture or horticulture. For instance, liquid carriers may include water, organic solvents, and mineral oils and vegetable oils. Suitable liquefied gaseous extenders or carriers are liquids which are gaseous at ambient temperature and under atmospheric pressure, for example aerosol propellants, such as butane, propane, nitrogen and carbon dioxide. A sticker is to be understood as meaning an additive or adjuvant to improve adhesive properties of the composition to the plant or part thereof. Suitable surfactants are emulsifiers, dispersants or wetting agents having ionic or nonionic properties, or mixtures of these surfactants. It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc. Stabilizers, such as low-temperature stabilizers, preservatives, antioxidants, light stabilizers or other agents which improve chemical and/or physical stability may also be present.

In certain embodiments, the agriculturally compatible carrier can be solid. Solid carriers can include but are not limited to clays, natural or synthetic silicates, silica, resins, waxes, solid fertilizers, a polymer, a granular mass, perlite, a perlite granule, peat, a peat pellet, soil, vermiculite, charcoal, sugar factory carbonation press mud, rice husk, carboxymethyl cellulose, fine sand, calcium carbonate, flour, alum, a starch, talc, polyvinyl pyrrolidone, or a combination thereof. In certain embodiments, the agriculturally compatible carrier can be a liquid. Liquid carriers can include but are not limited to water, alcohols, ketones, petroleum fractions, oils, aromatic or paraffinic hydrocarbons, chlorinated hydrocarbons, liquefied gases or a combination thereof. More particularly, the agriculturally compatible carrier can include a dispersant, a surfactant, an additive, a thickener, an anti-caking agent, residue breakdown, a composting formulation, a granular application, diatomaceous earth, a coloring agent, a stabilizer, a preservative, a polymer, a coating or a combination thereof. One of the ordinary skills in the art can readily determine the appropriate carrier to be used taking into consideration factors such as a particular bacterial strain, plant to which the inoculum is to be applied, type of soil, climate conditions, whether the inoculum is in liquid, solid or powder form, and the like. The additive can comprise an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutant dioate, a benzene acetonitrile derivative, a proteinaceous material, or a combination thereof. The proteinaceous material can include a milk product, wheat flour, soybean meal, blood, albumin, gelatin, or a combination thereof. The thickener can comprise a long chain alkylsulfonate of polyethylene glycol, polyoxyethylene oleate or a combination thereof. The surfactant can contain a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcohol, an alkyl phosphate, or a combination thereof. The anti-caking agent can include a sodium salt such as a sodium sulfite, a sodium sulfate, a sodium salt of monomethyl naphthalene sulfonate, or a combination thereof; or a calcium salt such as calcium carbonate, diatomaceous earth, or a combination thereof.

In certain embodiments, the bacterial strain(s) or bacterial population(s) can be supplied and/or applied in spray-dried or freeze-dried form.

In certain embodiments, the bacteria and optionally further plant-beneficial microorganism(s) may be administered in combination with one or more other non-active or active ingredients that are non-toxic thereto. Such other ingredients may be an oil, an emulsifier, a spreader, a cryoprotectant, a binder, a dispersant, a surfactant, a buffer, a tackifier, a stabilizer, a microbial stabilizer, a bactericide (e.g., effective against bacteria other than those administered), a fungicide, a complexing agent, a herbicide, a nematicide, an insecticide, a molluscicide, an algicide, a fertilizer, a micronutrient fertilizer material, a plant growth regulator, a rodenticide, a preservative, a polymer, a desiccant, a nutrient, an excipient, a wetting agent, a salt, or any combination thereof. In some embodiments the fertilizer is a liquid fertilizer. Liquid fertilizer can include without limitation, ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, hampene (chelated iron), dolomitic limestone, hydrate lime, calcium carbonate, diammonium phosphate, monoammonium phosphate, potassium nitrate, potassium bicarbonate, monopotassium phosphate, magnesium nitrate, magnesium sulfate, potassium sulfate, potassium chloride, sodium nitrates, magnesian limestone, magnesia, disodium dihydromolybdate, cobalt chlorid hexahydrate, nickel chloride hexahydrate, indole butyric acid, L-tryptophan, urea, urea-formaldehydes, urea ammonium nitrate, sulfur-coated urea, polymer-coated urea, isobutylidene diurea, K₂SO₄-2MgSO₄, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, peat moss, compost, green sand, cottonseed meal, feather meal, crab meal, fish emulsion or a combination thereof. In some embodiments the micronutrient fertilizer material can comprise boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate or a combination thereof. In some embodiments insecticide can include an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or a combination thereof. In some embodiments, the herbicide can comprise a chlorophenoxy compound, a nitrophenolic compound, a nitrocresolic compound, a dipyridyl compound, an acetamide, an aliphatic acide, an anilide, a benzamide, a benzoic acid, a benzoic acid derivative, anisic acid, an anisic acid derivative, a benzonitrile, benzothiadiazinone dioxide, a thiocarbamate, a carmabate, carbanilate, chloropyridinyl, a cyclohexenone derivative, a dinitroaminobenzene derivative, a fluorodinitrotoluidine compound, isoxazolidinone, nicotinic acide, isopropylamine, an isopropulamine derivative, oxadiazolinone, a phosphate, a phthalate, a picolinic acid compound, a triazine, a triazole, a uracil, a urea derivative, endothall, sodium chlorate, or a combination thereof. In some embodiments, the fungicide can comprise a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metlaxyl, thiamimefon, triforine, or a combination thereof.

In certain embodiments, the bacteria and optionally further plant-beneficial microorganism(s) may be co-administered and/or co-formulated with further biologicals or agrochemicals that stimulate plant growth and/or yield. In certain embodiments, particular strains may be selected on the basis of their compatibility with commonly used biologicals or agrochemicals. Plants, particularly agricultural plants, can be treated with a vast array of biologicals or agrochemicals. In some cases, particular strain may be selected to be compatible with biologicals or agrochemicals with complexing properties, to facilitate persistence of the strain in the plant. There also exist many complexing agents that do not penetrate the plant, at least at a concentration sufficient to interfere with the administered bacteria. Where a systemic complexing agent is used in the plant, compatibility of the strain to be inoculated with such agents may be an important variable to consider. In an embodiment, purified bacterial strains that are compatible with biologicals or agrochemicals can be used to inoculate plants, plant elements or growth media according to the methods described herein.

Bactericide-compatible strains can also be isolated by selection on liquid medium. The culture of strains can be plated on petri dishes without any forms of mutagenesis; alternatively, strains can be mutagenized using any means known in the art. For example, strain cultures can be exposed to UV light, gamma-irradiation, or chemical mutagens such as ethylmethanesulfonate (EMS), ethidium bromide (EtBr), dichlorvos (DDVP), methyl methane sulphonale (MMS), triethylphosphate (TEP), trimethylphosphate (TMP), nitrous acid, or DNA base analogs, prior to selection on bactericide comprising media. Alternatively or in addition, where the mechanism of action of a particular bactericide is known, the target gene can be specifically mutated (either by gene deletion, gene replacement, site-directed mutagenesis, etc.) to generate a strain that is resilient against that particular chemical. The above-described methods can be used to isolate strains that are compatible with both bacteriostatic and bactericidal compounds. The biological or agrochemical compatible strains generated can be detected in samples. For example, where a transgene was introduced to render the strain compatible with the biological(s) or agrochemical(s), the transgene can be used as a target gene for amplification and detection by PCR. In addition, where point mutations or deletions to a portion of a specific gene or a number of genes results in compatibility with the biological(s) or agrochemical(s), the unique point mutations can likewise be detected by PCR or other means known in the art. Such methods allow the detection of the strain even if it is no longer viable.

In certain embodiments, the composition is a liquid composition. The compositions may be a ready-to-use composition which can be administered or applied with a suitable apparatus, or the composition may be a concentrate or a concentrated formulation which is to be diluted in a solvent, such as water or an aqueous solution or buffer prior to use. In certain further embodiments, the composition is an aqueous composition. In certain embodiments, the composition is a sprayable liquid or a concentrate. In certain embodiments the composition is a spray, a sprayable liquid or a dip.

The compositions as intended herein can encompass an effective amount of the bacteria and optionally further plant-beneficial microorganism(s), i.e., an amount sufficient to elicit the desired outcome, such as in particular conferring control of or resistance against one or more plant pathogens in a plant compared to an untreated plant, or achieving one or more effects on one or more traits of agronomic importance in a plant (such as an increase in the biomass of a plant, or yield of a plant, or both biomass and yield of a plant) compared to an untreated plant, that is being sought by the user, in either a single or multiple doses, preferably in a single dose.

In certain embodiments, the composition comprises the bacteria at a concentration of at least about 10 CFU/ml. In certain embodiments, the composition comprises the bacteria at a concentration of at least about 10² CFU/ml. As used herein, a “colony forming unit” or “CFU” refers to a measure of viable microorganisms in a sample. A CFU is an individual viable cell capable of forming on a solid medium a visible colony whose individual cells are derived by cell division from one parental cell. The phrases “CFU”, “CFU/ml”, and “CFU/g” also encompass the reference to “spores”, “spores/ml” or “spores/g”, respectively, in case the microorganism lends itself to being administered in the form of spores.

In certain embodiments, the liquid composition comprises the bacteria at a concentration of at least about 10² CFU/ml. In certain embodiments, the liquid composition comprises the bacteria at a concentration of at least about 10³ CFU/ml, at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml.

In certain embodiments, the liquid composition comprises the bacteria at a concentration of from 1×10² CFU/ml to 1×10¹² CFU/ml, or from 1×10³ CFU/ml to 1×10¹¹ CFU/ml, or from 1×10³ CFU/ml to 1×10¹⁰ CFU/ml, or from 1×10⁴ CFU/ml to 1×10¹⁰ CFU/ml, or from 1×10⁵ CFU/ml to 1×10¹⁰ CFU/ml, or from 1×10⁶ CFU/ml to 1×10¹⁰ CFU/ml, or from 1×10⁶ CFU/ml to 1×10⁹ CFU/ml, or from 1×10⁷ CFU/ml to 1×10¹⁰ CFU/ml, or from 1×10⁷ CFU/ml to 1×10⁹ CFU/ml, or from 1×10⁸ CFU/ml to 1×10¹⁰ CFU/ml, or from 1×10⁸ CFU/ml to 1×10⁹ CFU/ml.

In certain embodiments, the composition is a non-liquid composition. In certain preferred embodiments, the composition may be a solid composition or a powdered composition. In certain preferred embodiments, the composition is a powder. The term “powder” refers to a dry, bulk solid composed of many very fine particles that may flow freely when shaken or tilted.

In certain embodiments, the non-liquid composition, such as the powder, comprises the bacteria at an amount of at least about 10 CFU/g. In certain embodiments, the non-liquid composition, such as the powder, comprises the bacteria at an amount of at least about 10² CFU/g. In certain embodiments, the non-liquid composition comprises the bacteria at an amount of at least about 10² CFU/g. In certain embodiments, the non-liquid composition comprises the bacteria at an amount of at least about 10³ CFU/g, at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g.

In certain embodiments, the non-liquid composition comprises the bacteria at an amount of from 1×10² CFU/g to 1×10¹² CFU/g, or from 1×10³ CFU/g to 1×10¹¹ CFU/g, or from 1×10³ CFU/g to 1×10¹⁰ CFU/g, or from 1×10⁴ CFU/g to 1×10¹⁰ CFU/g, or from 1×10⁵ CFU/g to 1×10¹⁰ CFU/g, or from 1×10⁶ CFU/g to 1×10¹⁰ CFU/g, or from 1×10⁶ CFU/g to 1×10⁹ CFU/g, or from 1×10⁷ CFU/g to 1×10¹⁰ CFU/g, or from 1×10⁷ CFU/g to 1×10⁹ CFU/g, or from 1×10⁸ CFU/g to 1×10¹⁰ CFU/g, or from 1×10⁸ CFU/g to 1×10⁹ CFU/g.

In certain embodiments, the composition may comprise bacteria of two or more bacterial strains as described in the present specification. In certain embodiments, the composition may comprise at least about 10² CFU/ml or at least about 10² CFU/g—such as the aforementioned more specific CFU/ml or CFU/g amount ranges—of bacteria of all the strains collectively or preferably of each of the strains individually and independently.

In certain embodiments, the composition comprises the one or more optional further plant-beneficial microorganism, such as cells or spores of one or more plant-beneficial microorganism strain, at a concentration or an amount, collectively or each individually and independently, of at least about 10² CFU/ml or 10² CFU/g, at least about 10³ CFU/ml or CFU/g, at least about 10⁴ CFU/ml or CFU/g, at least about 10⁵ CFU/ml or CFU/g, at least about 10⁶ CFU/ml or CFU/g, at least about 10⁷ CFU/ml or CFU/g, at least about 10⁸ CFU/ml or CFU/g, at least about 10⁹ CFU/ml or CFU/g, or at least about 10¹⁰ CFU/ml or CFU/g. More preferably, the composition comprises the one or more optional further plant-beneficial microorganism, such as cells or spores of one or more plant-beneficial microorganism strain, at a concentration or an amount, collectively or each individually and independently, of between 10³ to 10¹⁰ CFU/ml or CFU/g, between 10⁴ to 10¹⁰ CFU/ml or CFU/g, between 10⁵ to 10¹⁰ CFU/ml or CFU/g, between 10⁶ to 10¹⁰ CFU/ml or CFU/g, between 10⁶ to 10⁹ CFU/ml or CFU/g, between 10⁷ to 10⁹ CFU/ml or CFU/g, or between 10⁸ to 10⁹ CFU/ml or CFU/g.

In certain embodiments, the bacteria may be comprised by and administered as part of a bacterial population. A bacterial population may comprise bacteria of one or more bacterial strains as described in the present specification, such as of two, three or more strains as described in the present specification.

More generally, a bacterial population may comprise one or more, preferably two or more (e.g., 3, 4, 5, 6, 7, 8, 9, 10, 15, 20, 25 or more than 25) purified bacterial strains, wherein the strains may originate from different families of bacteria, or different genera of bacteria, or from the same genera but different species of bacteria. The taxonomically different bacterial strains can be obtained from the same cultivar of plant, different cultivars of the same plant, or different species of the same type of plant. The bacterial strains can be obtained from the soil wherein the plant is grown. In an embodiment in which one or more, preferably two or more purified bacterial strains are used, each of the bacterial strains can have different properties or activities, e.g., produce different metabolites, produce different enzyme, confer different beneficial traits, confer additional or complementary pathogen control or resistance in the plant, etc.

In certain embodiments, the bacterial population or composition may comprise bacteria of the one or more bacterial strain as described in the present specification, and optionally one or more additional bacterial strain. In certain embodiments, the bacteria of the one or more bacterial strain as described herein may collectively constitute at least about 1% by CFU of all viable bacteria constituting the bacterial population or composition, such as at least about 2%, at least about 5%, at least about 10%, preferably at least about 20%, such as at least about 30%, or at least about 40%, more preferably at least about 50%, such as at least about 60%, at least about 70%, at least about 80%, at least about 90%, at least about 95%, at least about 99%, or even 100% by CFU of all viable bacteria constituting the bacterial population or composition.

Where the bacterial population or composition comprises bacteria of two or more bacterial strains as described in the present specification, they may in certain embodiments be included in the population or composition in unequal amounts or preferably in about equal amounts. By means of an example and without limitation, the bacteria of each of the two or more bacterial strains as described herein may, each independently, constitute at least about 1% by CFU of all viable bacteria constituting the bacterial population or composition, such as at least about 2%, at least about 5%, at least about 10%, preferably at least about 20%, such as at least about 30%, or at least about 40%, more preferably at least about 50%, such as at least about 60%, at least about 70%, at least about 80%, at least about 90%, at least about 95%, or at least about 99% of all viable bacteria constituting the bacterial population or composition (where the sum of these amounts would exceed 100%, it shall be understood that the sum is capped at 100%).

Where the bacterial population or composition comprises bacteria of the one or more bacterial strain as described in the present specification and bacteria of one or more additional bacterial strain, each bacterial strain may in certain embodiments be included in the population or composition in unequal amounts or preferably in about equal amounts. By means of an example and without limitation, the bacteria of each of the bacterial strains may, each independently, constitute at least about 1% by CFU of all viable bacteria constituting the bacterial population or composition, such as at least about 2%, at least about 5%, at least about 10%, preferably at least about 20%, such as at least about 30%, or at least about 40%, more preferably at least about 50%, such as at least about 60%, at least about 70%, at least about 80%, at least about 90%, at least about 95%, or at least about 99% of all viable bacteria constituting the bacterial population or composition (where the sum of these amounts would exceed 100%, it shall be understood that the sum is capped at 100%).

In certain embodiments, the concentration or amount of each isolated bacterial strain in the bacterial population or composition may be at least about 10² CFU/ml or CFU/g, at least about 10³ CFU/ml or CFU/g, at least about 10⁴ CFU/ml or CFU/g, at least about 10⁵ CFU/ml or CFU/g, at least about 10⁶ CFU/ml or CFU/g, at least about 10⁷ CFU/ml or CFU/g, at least about 10⁸ CFU/ml or CFU/g, at least about 10⁹ CFU/ml or CFU/g, or at least about 10¹⁰ CFU/ml or CFU/g. More preferably, the concentration or amount of each isolated bacterial strain in the bacterial population or composition may be between 10³ to 10¹⁰ CFU/ml or CFU/g, between 10⁴ to 10¹⁰ CFU/ml or CFU/g, between 10⁵ to 10¹⁰ CFU/ml or CFU/g, between 10⁶ to 10¹⁰ CFU/ml or CFU/g, between 10⁶ to 10⁹ CFU/ml or CFU/g, between 10⁷ to 10⁹ CFU/ml or CFU/g, or between 10⁸ to 10⁹ CFU/ml or CFU/g.

Also provided in an aspect is a bacterial population, such as in accordance with the aforementioned explanations, comprising one or more bacterial strains selected from the group consisting of the strain deposited under the Budapest Treaty at the Polish Collection of Microorganisms (PCM) on 11 Feb. 2022 under Accession No. B/00366, or a functional mutant thereof; the strain deposited under the Budapest Treaty at the Polish Collection of Microorganisms (PCM) on 26 Nov. 2021 under Accession No. B/00364, or a functional mutant thereof; the strain deposited under the Budapest Treaty at the Polish Collection of Microorganisms (PCM) on 2 Nov. 2022 under Accession No. B/00435, or a functional mutant thereof; the strain deposited under the Budapest Treaty at the Polish Collection of Microorganisms (PCM) on 2 Nov. 2022 under Accession No. B/00436, or a functional mutant thereof; the strain deposited under the Budapest Treaty at the Polish Collection of Microorganisms (PCM) on 2 Nov. 2022 under Accession No. B/00437, or a functional mutant thereof; the strain deposited under the Budapest Treaty at the Polish Collection of Microorganisms (PCM) on 2 Nov. 2022 under Accession No. B/00438, or a functional mutant thereof; and combinations thereof.

In some embodiments, the bacterial population comprises one or more bacterial strains selected from the group consisting of the strain deposited under the Budapest Treaty at the Polish Collection of Microorganisms (PCM) on 11 Feb. 2022 under Accession No. B/00366, the strain deposited under the Budapest Treaty at the Polish Collection of Microorganisms (PCM) on 26 Nov. 2021 under Accession No. B/00364, the strain deposited under the Budapest Treaty at the Polish Collection of Microorganisms (PCM) on 2 Nov. 2022 under Accession No. B/00435, the strain deposited under the Budapest Treaty at the Polish Collection of Microorganisms (PCM) on 2 Nov. 2022 under Accession No. B/00436, the strain deposited under the Budapest Treaty at the Polish Collection of Microorganisms (PCM) on 2 Nov. 2022 under Accession No. B/00437, the strain deposited under the Budapest Treaty at the Polish Collection of Microorganisms (PCM) on 2 Nov. 2022 under Accession No. B/00438, and combinations thereof.

Preferably, the bacterial population comprises the bacterial strain with Accession no. B/00366.

Also provided in an aspect is an agricultural active composition, such as in accordance with the aforementioned explanations, comprising one or more bacterial strains selected from the group consisting of the strain deposited under the Budapest Treaty at the Polish Collection of Microorganisms (PCM) on 11 Feb. 2022 under Accession No. B/00366, or a functional mutant thereof; the strain deposited under the Budapest Treaty at the Polish Collection of Microorganisms (PCM) on 26 Nov. 2021 under Accession No. B/00364, or a functional mutant thereof; the strain deposited under the Budapest Treaty at the Polish Collection of Microorganisms (PCM) on 2 Nov. 2022 under Accession No. B/00435, or a functional mutant thereof; the strain deposited under the Budapest Treaty at the Polish Collection of Microorganisms (PCM) on 2 Nov. 2022 under Accession No. B/00436, or a functional mutant thereof; the strain deposited under the Budapest Treaty at the Polish Collection of Microorganisms (PCM) on 2 Nov. 2022 under Accession No. B/00437, or a functional mutant thereof; the strain deposited under the Budapest Treaty at the Polish Collection of Microorganisms (PCM) on 2 Nov. 2022 under Accession No. B/00438, or a functional mutant thereof; and combinations thereof.

In some embodiments, the agricultural active composition comprises one or more bacterial strains selected from the group consisting of the strain deposited under the Budapest Treaty at the Polish Collection of Microorganisms (PCM) on 11 Feb. 2022 under Accession No. B/00366, the strain deposited under the Budapest Treaty at the Polish Collection of Microorganisms (PCM) on 26 Nov. 2021 under Accession No. B/00364, the strain deposited under the Budapest Treaty at the Polish Collection of Microorganisms (PCM) on 2 Nov. 2022 under Accession No. B/00435, the strain deposited under the Budapest Treaty at the Polish Collection of Microorganisms (PCM) on 2 Nov. 2022 under Accession No. B/00436, the strain deposited under the Budapest Treaty at the Polish Collection of Microorganisms (PCM) on 2 Nov. 2022 under Accession No. B/00437, the strain deposited under the Budapest Treaty at the Polish Collection of Microorganisms (PCM) on 2 Nov. 2022 under Accession No. B/00438, and combinations thereof.

Preferably, the agricultural active composition comprises the bacterial strain with Accession no. B/00366.

The term “pathogen” or “plant pathogen” are well-known and broadly encompass organisms that cause harmful effects to the health and vigour of plants. Plant pathogens may include fungi, bacteria, viruses, insects, nematodes, etc. In certain embodiments, the pathogen is a bacterium or fungus. In certain embodiments, the pathogen is a bacterium. In certain particularly preferred embodiments, the pathogen is a fungus. In certain preferred embodiments, the pathogen is selected from the group consisting of Fusarium spp., Phytophthora spp., Pythium spp., Rhizoctonia spp., Puccinia spp., Mycosphaerella spp., Sclerotinia spp., Botrytis spp., Colletotrichum spp., Microdochium spp., Gaeumannomyces spp., Tapesia spp., Ustilago spp., Ramularia spp., Blumeria spp., Podosphaera spp., Alternaria spp., Monilinia spp., Penicillium spp. In a further preferred embodiment, the pathogen is Fusarium spp. In another preferred embodiment, the pathogen is Fusarium graminearum, also referred to as Gibberella zeae. In a further preferred embodiment, the pathogen is Puccinia spp. In another preferred embodiment, the pathogen is Puccinia striiformis var. tritici. In a further preferred embodiment, the pathogen is Mycrosphaerella spp. In another preferred embodiment, the pathogen is Zymoseptoria tritici, also referred to as Mycrosphaerella graminicola or Septoria tritici. In a further preferred embodiment, the pathogen is Podosphaera spp. In another preferred embodiment, the pathogen is Podosphaera xanthii.

Without limitation, Fusarium spp. include i.a. F. graminearum, F. pseudograminearum, and F. oxysporum), Phytophthora spp. include i.a. P. infestans, Rhizoctonia spp. include i.a. R. solani, Mycosphaerella spp. include i.a. M. graminicola also named Zymoseptoria tritici, Sclerotinia spp. include i.a. S. sclerotiorum, S. minor, and S. trifoliorum, Botrytis spp. include i.a. B. cinerea, Colletotrichum spp. include i.a. C. graminicola, Microdochium spp. include i.a. M. nivale, Gaeumannomyces spp. include i.a. G. graminis, Tapesia spp. include i.a. T. yallundae, Ramularia spp. include i.a. R. collo-cygni, Blumeria spp. include i.a. e.g. B. graminis, Podosphaera spp. include i.a. P. xanthii, Alternaria spp. include i.a. A. alternata, Monilinia spp. include i.a. M. fructigena, Penicillium spp. include i.a. P. digitatum, P. italicum, and P. expansum.

These pathogens are causal agents of known plant diseases. A plant disease may also be due to a combined infection by two or more phytopathogenic microorganisms, such as those above. The products and methods as taught herein may effectively control one or more pathogens in plants. In certain embodiments, the pathogen control or resistance afforded by the bacterial strain(s) disclosed herein may be broad-spectrum pathogen control or resistance.

Bacterial strains, combinations, populations, and compositions as discussed throughout the present specification can be employed in plants cultivation, in particular to facilitate pathogen control or resistance in plants, such as broad-spectrum pathogen control or resistance in plants. In certain embodiments, a treatment may be preventative, whereby the treatment inhibits or prevents a subsequent pathogen infection. In other embodiments, a treatment may be curative, once a plant has already been infected with a pathogen, and the treatment is applied to control or inhibit the growth and spreading of the pathogen, an possibly to reduce or eradicate the pathogen infection. Certain embodiments foresee a combination of preventative and curative treatments. Hence, the treatment can also allow the plant growth and/or yield to be improved, compared to an untreated reference plant exposed to the pathogen. In certain embodiments, despite the pathogen, the treated plant can display plant growth and/or yield at least comparable to reference plants not affected by the pathogen.

The terms “plant” or “plant element” as used herein encompasses whole plants, ancestors and progeny of the plants and plant parts, including seeds, shoots, stems, leaves, roots (including tubers), flowers, and tissues and organs. The terms “plant” or “plant element” also refer to plant cells, suspension cultures, callus tissue, embryos, meristematic regions, gametophytes, sporophytes, pollen and microspores. Hence, when the term “plant” or “plant element” is used herein, the term is intended to encompass “a plant, part thereof, or plant cell”. The term “plant cell” may encompass a non-propagating plant cell.

The phrases “part of a plant” or “plant part” as used herein refer to any one or more portions of a plant, such as to any one or more of the seeds, shoots, stems, leaves, roots (including tubers), flowers, and tissues and organs of a plant, such as for example meristematic tissue, ground tissue, vascular tissue, dermal tissue, etc. In addition, a “plant part” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keikis, or bud.

In certain embodiments, the part of a plant may be any one or more of the seeds, shoots, stems, leaves, roots (including tubers), or flowers. In certain embodiments, the part of a plant may be the seeds. In certain embodiments, the part of a plant may be the shoots, stems, or leaves. In certain embodiments, the part of a plant may be the roots (including tubers). In certain embodiments, the part of a plant may be tissues or organs of a plant.

In certain embodiments, the seeds, shoots, stems, leaves, roots (including tubers), flowers, tissues or organs of the plant, when treated according to the methods as taught herein, may be attached to (e.g., growing on) the whole plant. In certain embodiments, the seeds, shoots, stems, leaves, roots (including tubers), flowers, tissues or organs of the plant, when treated according to the methods as taught herein, may be detached from (e.g., not growing on) the whole plant. For instance, seeds may be detached from (e.g., not growing on) the whole plant when treated according to the methods as taught herein.

In some embodiments, plants may include wild plants and domesticated varieties. In certain embodiments, plants and plant parts may be developed by any technique, including but not limited to directed evolution, selection, marker assisted selection, hybridization, outcrossing, backcrossing, in-breeding, polyploidization, reverse breeding, doubled haploids, induced mutation, other genetic or epigenetic modifications, and combinations thereof.

The phrases “locus of a plant” or “locus of growth of a plant” as used herein refers to an area in close proximity of a plant (including parts thereof such as a seed). For instance, the locus of a plant may be a circular area around the plant, e.g., around the stem of a plant or around a seed, such as a circular area having a diameter of at most 1 meter, for instance at most 50 centimeters (cm), at most 40 cm, at most 30 cm, at most 20 cm, at most 10 cm, or at most 5 cm, around the plant, e.g., around the stem of a plant or around a seed. The locus of growth may include the growth medium (e.g., soil, hydroponic medium, or hydroculture medium) for cultivating the plant.

The reference to plants includes any plants. In certain embodiments, the plants may be an angiosperm. Particularly preferred are agricultural plants. The terms “agricultural plants”, “crops” or “plants of agronomic importance” as used herein include plants that are cultivated by humans for but not limited to food, feed, fiber, fuel, gardening, and/or industrial purposes.

In certain embodiments, the plant is a monocotyledon. The terms “monocotyledon” or “monocot” refer to flowering plants (angiosperms) whose seeds typically contain only one embryonic leaf or cotyledon.

In certain preferred embodiments, the plant is a cereal plant. The terms “cereal” or “cereal plant” refer to any grass cultivated for the edible components of its grain (caryopsis), composed of the endosperm, germ, and bran.

In certain preferred embodiments, the plant is selected from the group consisting of wheat, maize, barley, rice, millet, rye, triticale, sorghum, emmer, spelt, einkorn, teff, milo, and oats. In certain particularly preferred embodiments, the plant is wheat or maize. In certain particularly preferred embodiments, the plant is wheat (Triticum aestivum and related varieties).

In other embodiments, the plant is a dicotyledon. The terms “dicotyledon” or “dicot” refer to flowering plants (angiosperms) whose seeds typically contain two embryonic leaves or cotyledons. In certain particularly preferred embodiments, the plant is a cucurbit, such as a cucumber plant. In certain embodiments, the products and methods disclosed herein may be particularly useful for monocotyledonous and dicotyledonous plants including fodder or forage legumes, ornamental plants, food crops, trees or shrubs, such as plants selected from the list comprising or consisting of Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Axonopus spp., Bambusa spp., Benincasa hispida, Bertholletia excelsea, Beta vulgaris, Brassica spp. (e.g. Brassica napus), Bouteloua spp., Brassica spp., Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Coix spp., Colocasia esculenta, Cola spp., Corchorus sp., Coriandrum sativum, Corylus spp., Crataegus spp., Crocus sativus, Cucurbita spp., Cucumis spp., Cynara spp., Cynodon spp., Dactylis spp., Daucus carota, Desmodium spp., Dimocarpus longan, Dioscorea spp., Diospyros spp., Echinochloa spp., Elaeis spp. (e.g. Elaeis guineensis, Elaeis oleifera), Eleusine coracana, Eragrostis spp. (e.g. Eragrostis tef), Eremochloa spp., Erianthus spp., Eriobotrya japonica, Eucalyptus spp., Eugenia uniflora, Fagopyrum spp., Fagus spp., Festuca spp. (e.g. Festuca arundinacea), Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca spp. (e.g. Lactuca sativa), Lathyrus spp., Lens culinaris, Linum usitatissimum, Litchi chinensis, Lolium spp., Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Paspalum spp., Panicum spp. (e.g. Panicum miliaceum, Panicum virgatum), Passiflora edulis, Pastinaca sativa, Pennisetum spp., Persea spp., Petroselinum crispum, Phalaris arundinacea, Phaseolus spp., Phleum spp. (e.g. Phleum pretense), Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale spp. (e.g. Secale cereal), Sesamum spp., Sinapis sp., Solanum spp. (e.g. Solanum tuberosum, Solanum integrifolium, Solanum lycopersicum), Sorghum spp. (e.g. Sorghum bicolor), Spinacia spp., Stenotaphrum spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum, Triticum vulgare), Topaeolum spp. (e.g. Tropaeolum minus, Tropaeolum majus), Vaccinium spp., Vicia spp., Vigna spp., Viola odorata, Vitis spp., Zea mays, Zizania palustris, Ziziphus spp., Zoysia spp., amongst others; including the progenies and hybrids between the above.

In certain preferred embodiments, the plant, more in particular the agricultural plant, as envisaged herein is wheat (Triticum aestivum and related varieties), rye (Secale cereale and related varieties), or barley (Hordeum vulgare and related varieties).

In certain particularly preferred embodiments, the plant, more in particular the agricultural plant, as envisaged herein is wheat (Triticum aestivum and related varieties).

In certain other preferred embodiments, the plant, more in particular the agricultural plant, as envisaged herein belongs to Cucurbitaceae. In certain preferred embodiments, the plant may be Cucurbita spp., Citrullus spp., or Cucumis spp., such as particularly preferably cucumber (C. sativus and related varieties).

The plant may be a non-modified plant or a modified plant. As used herein, a plant may be “modified” when it comprises an artificially introduced genetic or epigenetic “modification”. In some embodiments, the modification is introduced by a genome engineering technology. In some embodiments, the modification is introduced by a targeted nuclease. In some embodiments, targeted nucleases include, but are not limited to, transcription activator-like effector nuclease (TALEN), zinc finger nuclease (ZNF), clustered regulatory interspaced short palindromic repeats (CRISPR), CRISPR/Cas9, CRISPR/CPFL and combinations thereof. In some embodiments, the modification is an epigenetic modification. In some embodiments, the modification is introduced by treatment with a DNA methyltransferase inhibitor such as 5-azacytidine, or a histone deacetylase inhibitor such as 2-amino-7-methoxy-3H-phenoxazin-3-one. In some embodiments, the modification is introduced via tissue culture. In some embodiments, a modified plant may comprise a transgene. In certain embodiments, the plant may be a non-transgenic plant or a transgenic plant.

In certain preferred embodiments, the plant may be a non-transgenic plant or a transgenic plant. The terms “recombinant”, “transgenic” or “transgene” as used herein, for example with regard to a plant, refer to those plants brought about by recombinant methods in which a nucleic acid sequence and/or genetic control sequence(s) which are operably linked to the nucleic acid sequence are not located in their natural genetic environment. The natural genetic environment is understood as meaning the natural genomic or chromosomal locus in the original plant. A naturally occurring nucleic acid sequence (e.g., a naturally occurring combination of the native promoter of a nucleic acid sequence, the corresponding native nucleic acid sequence encoding a protein, and the native transcription termination sequence of a nucleic acid sequence) becomes a recombinant nucleic acid when this nucleic acid is not integrated in the natural genetic environment but in a different genetic environment as a result of an isolation of said nucleic acid from its natural genetic environment and re-insertion at a different genetic environment.

In certain embodiments, the plant may be free of disease and/or pathogen pressure and/or pest organisms. In other embodiments, the plant may be affected with disease and/or pathogen pressure and/or pest organisms.

The products, methods and uses as taught herein can provide for advantages in plants treated therewith relative to untreated plants. An “untreated plant” refers to a plant of the same species as (e.g., which is isogenic to or genetically identical to) and grown under substantially the same conditions as (e.g., for the same amount of time, in the same climate, and cultivated according to the same methods using the same materials, with pathogen control or resistance and other characteristics being measured according to the same methods) a plant which has been administered the bacterial strain(s) (for reasons of brevity, the mention of bacterial strain(s) henceforth encompasses the one or more bacterial strain as envisaged herein, as well as the bacterial strain combinations and bacterial populations as disclosed herein, as well as the compositions comprising these, insofar the context does not indicate otherwise; these may also contain the optional further plant-beneficial microorganism(s)), except that the untreated plant has not been administered said bacterial strain(s) to the plant, a part thereof, a seed for growing the plant, or locus of the plant. The term may be used synonymously to “reference plant” or “reference”, a plant genetically identical to and handled in substantially identical ways to a treated plant, with the exception of the treatment under investigation, and which thus offers a meaningful and informative control for detecting the effects of said treatment. A treated plant and a control reference plant can thus be exposed to substantially the same environmental conditions. By means of an example, the treated plant and reference plant can both be observed under substantially identical conditions of drought stress, or the treated plant and reference plant can both be observed under substantially identical conditions of no drought stress.

The term “untreated seed” refers to a seed of the same species as (e.g., which is isogenic to or genetically identical to) and obtained under substantially the same conditions (e.g., plants from which the seeds are obtained are grown for the same amount of time, in the same climate, and cultivated according to the same methods using the same materials, seeds are stored under the same conditions) as a seed which has been administered the bacterial strain(s), except that the untreated seed has not been administered said bacterial strain(s).

The term “pathogen control” or “pathogen resistance” is intended to broadly encompass any feature that relates in some way to the absence or reduction of plant disease features, or reduced effects caused by a pathogen. The feature may relate to or be observable with respect to an individual plant or to a population of plants. Examples of such features include, without limitation, absence of the disease, reduced disease severity, absence of necrosis, reduced necrosis, etc. Present products and methods can provide for pathogen control or resistance in vitro as well as in planta.

The reference to a reduction encompasses any qualitative or quantitative change or modification in a plant disease feature that is industrially beneficial, in particular in the context of agriculture. To the extent a plant disease feature is quantifiable, a reduction may be synonymous to a reduction in that quantity, depending on the nature of the plant disease feature. By means of an example and without limitation, a reduction may be desired in quantifiable features such as the affected leave area, the presence or the amount of necrosis, or the presence of white spikes on the leaves, etc. In certain embodiments, the plant disease feature comprises or is disease severity, necrosis, the area of affected leaves of the plant, the presence of white spikes on the leaves.

The term “decrease” as used herein is intended to be synonymous with terms such as “downregulate”, “inhibit”, or “reduce”. An decrease in a plant disease feature, such as disease severity or necrosis, can be in the whole plant or in any part thereof such as aboveground (harvestable) parts, vegetative biomass, roots, fruits, or seeds. Any extent or degree of such decrease is contemplated herein, in particular any agronomically meaningful decrease. Typically, the term may in appropriate contexts, such as in experimental or agricultural contexts, denote a statistically significant decrease relative to a reference. The skilled person is able to select such a reference, as also discussed elsewhere in this specification. For example, such decrease may fall outside of error margins for the reference (as expressed, for example, by standard deviation or standard error, or by a predetermined multiple thereof, e.g., ±1×SD or ±2×SD, or ±1×SE or ±2×SE). Accordingly, while the respective decreases may be observable at the level of individual plants, they are more usefully evaluated by comparing relevant population characteristics, such as average or median values of the respective traits, obtained using sample sizes of treated vs. untreated plants that allow for statistically meaningful conclusions, such as for example shown in the Examples section.

In certain embodiments, the plant disease feature, such as disease severity or necrosis, in a plant may each independently be decreased by at least about 1% relative to (i.e., compared with) (i.e., the disease severity or necrosis may be 0.99-fold or less) the disease severity or necrosis of an untreated plant, such as preferably decreased by at least about 2% (i.e., 0.98-fold or less), by at least about 3% (i.e., 0.97-fold or less), by at least about 5% (i.e., 0.95-fold or less), by at least about 10% (i.e., 0.90-fold or less), by at least about 15% (i.e., 0.85-fold or less), by at least about 20% (i.e., 0.80-fold or less), such as decreased by at least about 25% (i.e., 0.75-fold or less), by at least about 30% (i.e., 0.70-fold or less), by at least about 35% (i.e., 0.65-fold or less), by at least about 40% (i.e., 0.60-fold or less), by at least about 45% (i.e., 0.50-fold or less), by at least about 50% (i.e., 0.50-fold or less), by at least about 60% (i.e., 0.40-fold or less), by at least about 70% (i.e., 0.30-fold or less), by at least about 80% (i.e., 0.20-fold or less), by at least about 90% (i.e., 0.10-fold or less), or even by 100%. For example, the disease severity or necrosis of a plant may be decreased by between 2% and 5%, between 5% and 10%, between 10% and 15%, between 15% and 20%, between 20% and 30%, between 30% and 40%, or between 40% and 50%, or between 50% and 60%, or between 60% and 70%, or between 70% and 80%, or between 80% and 90%, or between 90% and 100% relative to the disease severity or necrosis of an untreated plant. Such reduced disease severity or necrosis may advantageously reduce or even abolish the need to treat the plants with anti-pathogen agents such as pesticides in the field and thereby advantageously leads to more sustainable agriculture. As said above, these percentages or fold decreases may conveniently reflect the relationships between averages for the respective traits in the treated vs. untreated populations, as determined by evaluating representative population samples.

The term “plant growth feature” is intended to broadly encompass any feature that relates in some way to plant growth. The feature may relate to or be observable with respect to an individual plant or to a population of plants. Examples of such features include, without limitation, plant wet or dry biomass, plant height, plant size, emergence %, emergence date, canopy cover, flowering status, seed yield, grain yield, fruit yield, number of tillers per plant, shoot length, root length, root architecture, seed weight, senescence, stay-green, number of mature plant reproductive elements per plant, visual appearance, etc.

In certain embodiments, the seed or grain yield of the plant may be increased, such as increased by the aforementioned percentages or fold change.

As set forth elsewhere in the specification, the bacterial strain(s) can thus be administered to the plant, a part of the plant, a seed for growing the plant, or locus of the plant in an amount effective to produce pathogen control or resistance in the plant compared to an untreated plant.

The phrase “administering” generally refers to man- and/or machine-driven or effected disposing, applying, delivering, or providing of a recited object, such as the bacterial strain(s), to a recipient entity, such as the plant, a part thereof, a seed for growing the plant, or locus of the plant. The bacterial strain(s) as taught herein may be administered by any known method wherein all or part of the plant is treated, such as by root, seed, or foliar inoculation. For example, the administration can be to the aerial portions of a plant, such as the leaves and stem, to the roots of the plant, to the seed of the plant prior to planting the seed in soil, or to the soil or plant growth medium surrounding the plant or plant seed. Application methods such as spraying, coating, covering, contacting, and/or immersion can be adopted. In certain embodiments, application may be to a surface, such as to the surface of growth medium (such as soil), plant, plant part, seeds, harvested crop, harvested seed crop, stored crops or crop parts. In certain embodiments, the administration may be to the plant, part thereof, or locus of the plant, present on the field. The terms “field” or “agricultural field” may be used interchangeably herein and refers to an area of land used for agricultural purposes such as cultivating crops, such as cultivating wheat plants or maize plants.

A non-limiting example concerns the spraying of an agricultural formulation comprising one or more purified bacterial strains or bacterial populations of embodiments of the current invention against Fusarium spp. by spraying the ears of a Triticum aestivum plants. In another non-limiting example, a curative or preventive treatment of Triticum aestivum plants against Puccinia spp. infections may be effected by spraying an agricultural formulation comprising the one or more purified bacterial strains or bacterial populations of embodiments of the current invention on the leaves of said plants. Another non-limiting example concerns the biocontrol of Fusarium spp., Blumeria spp, and/or Puccinia spp. on wheat, and Podosphaera xanthii on cucurbits, via foliar application of one or more purified bacterial strains or bacterial populations of embodiments of the current invention.

In certain embodiments, the bacterial strain(s) may be applied to the part of the plant, when forming part of the plant. For instance, they may be applied to the part of the plant, such as to the leaf, when the part of the plant, such as the leaf, is present on or attached to (e.g., is growing on) the plant.

In certain embodiments, the bacterial strain(s) may be applied to any one or more of the seeds, shoots, stems, leaves, roots (including tubers), flowers, tissues, or organs of a plant. In certain embodiments, the bacterial strain(s) may be applied to (e.g., sprayed on) the whole of the aboveground part of the plant. Accordingly, in certain embodiments, the bacterial strain(s) may be applied to (e.g., sprayed on) any one or more of the shoots, stems, leaves, or flowers of the plant. Preferably, the bacterial strain(s) may be applied to (e.g., sprayed on) any one or more of the shoots, leaves, or flowers of the plant. In certain embodiments, the bacterial strain(s) may be applied to (e.g., sprayed on) the shoots of the plant.

In certain embodiments, the bacterial strain(s) may be administered to the locus of the plant, such as by inoculating the growth medium. Hence, in certain embodiments, the method comprises inoculating soil or a plant growth medium with the bacteria and growing the plant in said soil or medium.

The terms “growth medium” or “plant growth medium” as used herein refer to a substrate or medium for culturing plants. In certain embodiments, the plant may be grown in soil or in a growth medium, including soil-less culture. Growing media provide rooting environment for plants and are used in professional horticulture for fruity vegetables, pot plants, young plant production, tree nursery stock, cut flowers, bedding plants and soft fruits. Commonly known as “potting soil” or “substrate”, growing media are also used in the hobby market. The range of growing media constituents used includes peat, coir pith, wood fibers, bark, composted materials, i.e. green waste, and bark. Mineral constituents like perlite, pumice, clay and vermiculite are also used. Growing media are often formulated from a blend of such raw materials, usually enriched with fertilizers, lime and sometimes biological additives in order to achieve the correct balance of physical, chemical and biological properties for the plants to be grown. Having the right growing media mix is as important for an optimal plant growth as water and fertilisers. In embodiments, the growth medium may be soil, green waste compost, peat (black and white), coco-coir, coco-fibres and coco-chips, wood fibres, miscanthus, (composted) bark, perlite, clay and other biobased materials, a soil-mimicking substrate such as mineral lava or basalt substrate, textile, or a soil-less substrate, such a stone wool. In certain embodiments, the grown medium may be sand, gravel, polysaccharide, mulch, peat moss, straw, logs, clay, or a combination thereof. In embodiments, the plant growth medium may also include a hydroculture system or an in vitro culture system. Hence, in certain embodiments, the plant growth medium is a hydroponic medium or a hydroculture medium. The skilled person understands that different types of growth media may be used for growing different types of plants. Inoculating a plant growth medium can be performed, by way of example using a liquid, or a solid product, such as a powder, a granule, a pellet and as a blend together with the fertilizer.

The term “soil-less culture” is commonly denotes the cultivation of plants in systems without soil “in situ”. The methods of growing plants without soil fall into two general categories. Liquid culture (true hydroponics), where the nutrient solution is recirculated after re-aeration and adjustment of the acidity and nutrient levels, like the nutrient film technique (NFT); and aggregate culture, where the nutrient solution is supplied to plants via an irrigation system through the growing medium, and excess fertigation solution is allowed to drain away or the fertigation solution is recirculated.

Overview of the different soil-less culture systems is given inter alia by Olympios 1999 (Overview of soilless culture: advantages, constraints, and perspectives, Cahiers Options Méditerranéennes, n. 31, p. 307-324). Hydroculture, also encompassing hydroponics, is the growing of plants in a soil-less medium or an aquatic based environment, while in vitro culture system refers to the growing of plants or explants on or in a recipient with synthetic medium, in sterile conditions, in a controlled environment and in reduced space. In controlled environment agriculture, the recent development of state-of-the-art vertical farms allows maximizing plant growth in a resource use efficient way (water, CO2, fertilizer, energy). Plant factories with artificial lighting can tap into new markets inaccessible to open-field production and conventional greenhouses by locally producing leafy greens, herbs, medicinal plants, and transplants year-round for local consumption. Plant factories with artificial lighting utilize soilless culture methods. Soilless culture typically requires a plant growing medium that provides a proper physicochemical and biological environment for rooting and plant growth during the seedling stage. Explants refer to parts of a plant, from all the aerial part to isolated cells, as parts of leaves, of roots, seeds, bulbs, tubers, buds. The inoculation of the plant growth medium with the bacterial strain(s) may be performed before, during and/or after sowing or before, during and/or after the start of the plant growth cycle in case of hydroculture or in vitro culture. The inoculation can be performed once or multiple times during the plant growth cycle.

In certain embodiments, sprayable liquids may be applied by spraying the plant, part thereof, or locus of the plant by conventional spraying equipment as known in the art, such as airplanes, backpack sprayers, tractor mounted boom sprayers etc.

In certain embodiments, application of the bacterial strain(s) to the plant, part thereof, or locus of growth of the plant may be carried out directly or by action on their surroundings or habitat using customary treatment methods, for example by dipping, drenching, spraying, coating, atomizing, irrigating, evaporating, dusting, fogging, broadcasting, foaming, painting, spreading-on, watering (drenching) or drip irrigating. In embodiments, the method may comprise spraying, sprinkling, showering, spritzing, spreading in droplets, spattering; dispersing, diffusing, or douching the plant, part thereof, or locus of growth of the plant with the bacterial strain(s).

In certain embodiments, the bacteria of the one or more bacterial strain as taught herein may be applied to a locus where plants are or are to be grown, such as upon soil, such as upon a field or within a greenhouse, in an amount of from about 1×10⁸ CFU/hectare to about 1×10¹⁴ CFU/ha, such as from about 1×10⁹ CFU/hectare to about 1×10¹³ CFU/ha, or from about 1×10¹⁰ CFU/hectare to about 1×10¹² CFU/ha, preferably about 1×10¹¹ CFU/hectare

In certain embodiments, the application may be one-time (single) administration, repeated administration (i.e., more than one time administration) at the same or varying time intervals, or continuous administration. The bacterial strain(s) can be administered at any point in the life cycle of the plant (e.g., before or after germination). For example, administration can be to a plant's seed prior to planting the seed in soil and prior to germination. Alternatively, administration can be to the plant (e.g. a seedling), the seed of the plant, or the soil surrounding the plant after germination has occurred. Once treated with the bacterial strain(s), seeds can be planted in soil and cultivated using conventional methods for generating plant growth.

In certain embodiments, the bacterial strain(s) may be applied at a temperature (e.g., air temperature) in the range from −1° C. to 30° C. In embodiments, the application may be at a temperature in the range from 0° C. to 30° C., from 1° C. to 30° C., from 5° C. to 25° C., or from 10° C. to 20° C.

In certain embodiments, the bacterial strain(s) may be applied to the part of the plant, when not forming part of the plant. For instance, they may be applied to the part of the plant, such as to a seed for growing the plant, when the part of the plant, such as the seed, is not present on or is detached from (e.g., is not growing on) the plant. For instance, they may be applied to seeds after they have been harvested from (e.g. mechanically or manually separated from) the plant. In certain embodiments, the method may comprise administering the bacterial strain(s) to a seed of the plant, e.g., prior to planting the seed or with the seed at planting. Hence, in certain embodiments, the method comprises administering the bacterial strain(s) to a seed of the plant. In certain embodiments, the bacterial strain(s) are administered to the seed of the plant prior to planting the seed, or with the seed at planting, or after planting the seed and before germination of the seed. In certain embodiments, the purified bacterial strain(s) are capable of colonizing plants. Successful colonization can be confirmed by detecting the presence of the strain within the plant. For example, after applying the strain to the plant parts, high titers of the strain can be detected in the roots and shoots of the plants that germinate from said plant parts such as seeds. Detecting the presence of the strain inside the plant can be accomplished by measuring the viability of the strain after surface sterilization of the plant element or the plant: strain colonization results in an internal localization of the strain, rendering it resistant to conditions of surface sterilization. The presence and quantity of strain can also be established using other means known in the art, for example, immunofluorescence microscopy using microbe-specific antibodies, or fluorescence in situ hybridization. Alternatively, specific nucleic acid probes recognizing conserved sequences from an strain can be employed to amplify a region, for example by quantitative PCR, and correlated to CFUs by means of a standard curve.

Hence, in certain embodiments, microorganisms such as bacteria are said to colonize a plant, plant part, root or seed, when they can exist in relationship with a plant or plant part during at least part of either the plant's or the microorganism's life cycle. In certain embodiments, microorganisms such as bacteria are said to colonize a plant when they can be stably detected within the plant or plant part over a period time, such as one or more days, weeks, months or years. The compositions and methods described herein may in certain embodiments comprise one or a plurality of bacterial strains as taught herein and optionally one or more further plant-beneficial microorganism in amounts effective to colonize a plant.

In embodiments, the strains described herein may be capable of moving from one tissue type to another. For example, the detection and isolation of strains within the mature tissues of plants after treating the exterior of a plant part demonstrates their ability to move from the plant part into the vegetative tissues of a maturing plant. Therefore, in some embodiments, the population of bacterial strains is capable of moving from the plant element exterior into the vegetative tissues of a plant. In some embodiments, the strain that is disposed onto the plant element of a plant is capable, upon germination of the plant part into a vegetative state, of localizing to a different tissue of the plant. For example, strains can be capable of localizing to any one of the tissues in the plant, including: the root, adventitious root, seminal root, root hair, shoot, leaf, flower, ear, spike, spikelet, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In an embodiment, the strain is capable of localizing to the root and/or the root hair of the plant. In another embodiment, the strain is capable of localizing to the photosynthetic tissues, for example, leaves and shoots of the plant. In other cases, the strain is localized to the vascular tissues of the plant, for example, in the xylem and phloem. In still another embodiment, the strain is capable of localizing to the reproductive tissues (flower, pollen, pistil, ovaries, stamen, fruit, spike, spikelet) of the plant. In another embodiment, the strain is capable of localizing to the root, shoots, leaves and reproductive tissues of the plant. In still another embodiment, the strain colonizes a fruit or plant element tissue of the plant. In still another embodiment, the strain is able to colonize the plant such that it is present in the surface of the plant (i.e. its presence is detectably present on the plant exterior). In still other embodiments, the strain is capable of localizing to substantially all, or all, tissues of the plant. In some cases, strains are capable of replicating within the host plant and colonizing the plant.

In further embodiments, following administration of the bacterial strain(s), the plants are cultivated under conditions conducive to plant growth and development. In other words, the present methods may further comprise cultivating the plant under conditions conducive to plant growth and development.

In certain embodiments, the method comprises administering the bacterial strain(s) to a seed of the plant, a whole plant, or a seedling, and optionally cultivating the seed, whole plant, or seedling under conditions conducive to plant growth and development. In certain embodiments, the method comprises administering the bacterial strain(s) to a seed of the plant, and optionally cultivating the seed under conditions conducive to plant growth and development. Hence, in such latter embodiments, the plant is grown from a seed, in particular a seed planted in said soil or plant growth medium.

Cultivating the seed under conditions conducive to plant growth and development, may but need not include growth to maturity and/or regeneration.

In certain embodiments, the method may comprise further propagating the plant treated with the bacterial strain(s). Accordingly, the invention provides a method for conferring a pathogen control or resistance in a plant grown from a seed compared to a plant grown from an untreated seed, the method comprising administering the bacterial strain(s) to the seed for growing the plant. The invention also provides the use of the bacterial strain(s) for conferring a pathogen control or resistance in a plant grown from a seed compared to a plant grown from an untreated seed. Also provided herein is a method of treating a seed of a plant comprising inoculating the seed with bacteria of one or more bacterial strain as taught herein, such that the bacteria colonize a plant germinated from the inoculated seed and/or the soil or plant growth medium surrounding the growing plant, whereby the bacteria confer a pathogen control or resistance in the plant, compared to a plant germinated from an untreated seed. In certain embodiments, the seed is coated with the bacteria, incubated with the bacteria, or planted near the bacteria. In certain embodiments, the seed is further inoculated with one or more additional plant-beneficial microorganism as taught herein.

In certain embodiments, the bacteria of one or more bacterial strain as taught herein may be contacted with seeds in an amount of between about 1×10⁶ CFU/kg seeds to about 1×10¹² CFU/kg seed, such as between about 1×10⁷ CFU/kg seeds to about 1×10¹¹ CFU/kg seeds, or between about 1×10⁸ CFU/kg seeds to about 1×10¹⁰ CFU/kg seeds, or between about 1×10⁹ CFU/kg seeds to about 1×10¹⁰ CFU/kg seeds, preferably about 2.5×10⁹ CFU/kg.

In certain embodiments, the bacteria of one or more bacterial strain as taught herein may be coated on or present on or inoculated into seeds at a quantity of, on average, at least 10 CFU per seed, preferably at least 10⁰ CFU per seed, more preferably at least 500 CFU per seed, and even more preferably at least 1000 CFU per seed, such as between 1×10³ and 1×10⁵ CFU per seed, or between 1×10³ and 1×10⁴ CFU per seed, or for example at least 1×10⁴ CFU per seed, or 1×10⁵ CFU per seed or 1×10⁶ CFU per seed, such as between 1×10⁵ and 1×10⁷ CFU per seed, preferably about 1×10⁶ CFU per seed. Certain embodiments thus provide a plant seed comprising at least 10 CFU preferably at least 10⁰ CFU, more preferably at least 500 CFU, and even more preferably at least 1000 CFU, such as more preferably at least 1×10⁴ CFU, or 1×10⁵ CFU, or 1×10⁶ CFU, such as between 1×10⁵ and 1×10⁷ CFU, preferably about 1×10⁶ CFU, of the bacteria as taught herein. For example, the seed may be coated with the bacterial cells or the composition as taught herein.

In certain embodiments, the bacteria of one or more bacterial strain as taught herein can be cultured on a culture medium or can be adapted to culture on the culture medium. Said culture medium is sterile prior to being inoculated with the bacterial strain and comprises all nutrients for growth and maintenance of the strain on the culture medium. In addition, the culture medium can be in a solid, semi-solid or liquid form. The isolation, identification, and culturing of the microbes of present invention can be effected using standard microbiological techniques. Isolation can be effected by streaking the specimen on a solid medium (e.g., nutrient agar plates) to obtain a single colony, which is characterized by the phenotypic traits (e.g., Gram-positive/negative, capable of forming spores aerobically/anaerobically, cellular morphology, carbon source metabolism, acid/base production, enzyme secretion, metabolic secretions, etc.) and to reduce the likelihood of working with a culture which has become contaminated.

In certain embodiments, the bacteria of one or more bacterial strain as taught herein may be administered as a whole cell broth, comprising the bacteria as well as the medium in which the bacteria have been grown.

In some embodiments, the bacteria of one or more bacterial strains as taught herein may be administered as spores.

A further aspect provides a plant or part thereof treated with the bacterial strain(s). Also provided is a plant or part thereof heterologously disposed with the bacteria as taught herein. In certain embodiments, the plant part may be a seed, such as a seed coated with the bacteria or the composition. In certain embodiments, the plant or part thereof or a plant grown from said plant part can display increased vigor and biomass as compared to an untreated plant or part thereof.

Hence, also disclosed is a plant seed, preferably a crop plant seed (e.g., the seed of a wheat plant or maize plant), treated with, such as coated with, the bacterial strain(s), e.g., such that all or part of the seed has a coating or film comprising the bacteria. In certain embodiments, the plant grown from the treated seed can display increased vigor and biomass as compared to a plant grown from an untreated seed.

Further provided is a plant grown from a plant or part thereof (e.g., seed) treated with the bacterial strain(s).

In an embodiment, bacteria of the one or more strain as taught herein may, collectively or preferably each of the strains individually and independently, be present in an amount of at least about 10² CFU per treated plant or part thereof. In an embodiment, bacteria of the one or more strain as taught herein may, collectively or preferably each of the strains individually and independently, be present in an amount of at least about 10² CFU per plant grown from the treated plant or part thereof such as from a treated seed.

Preferably, bacteria of the one or more strain as taught herein may, collectively or preferably each of the strains individually and independently, be present on the plant or part thereof in an amount effective to be detectable within a target tissue of the mature plant selected from a fruit, a seed, a leaf, or a root, or portion thereof. For example, in an amount of at least about 10⁰ CFU, between 100 and 200 CFU, at least about 200 CFU, between 200 and 300 CFU, at least about 300 CFU, between 300 and 400 CFU, at least about 500 CFU, between 500 and 1,000 CFU, at least about 1,000 CFU, between 1,000 and 3,000 CFU, at least about 3,000 CFU, between 3,000 and 10,000 CFU, at least about 10,000 CFU, between 10,000 and 30,000 CFU, at least about 30,000 CFU, between 30,000 and 100,000 CFU, at least about 10⁵ CFU, between 10⁵ and 10⁶ CFU, at least about 10⁶ CFU or more in the mature plant.

In certain embodiments, the plant or part thereof, such as a seed, treated with (e.g. coated with) the bacteria as taught herein may be shelf-stable. The bacterial strain may be shelf-stable, where at least 0.01%, of the CFUs are viable after storage in desiccated form (i.e., moisture content of 30% or less) for 1, 2, 3, 4, 5, 6, 7, 8, 9, 10 or greater than 10 weeks at 4° C. or at room temperature.

Optionally, a shelf-stable composition comprising the bacteria may be in a dry composition, a powder composition, or a lyophilized composition. For example, the microbes may be spray-dried or freeze-dried. Drying processes for microorganisms are known for a person skilled in the art. In some embodiments, adjuvants may be added that provide for a pertinent shelf-life. In an embodiment, the composition may be formulated to provide stability for the strains. In an embodiment, the plant or part thereof, such as a seed, treated with (e.g. coated with) the bacteria may be substantially stable at temperatures between about −20° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years. In another embodiment, the plant or part thereof, such as a seed, treated with (e.g. coated with) the bacteria may be substantially stable at temperatures between about 4° C. and about 37° C. for at least about 5, 10, 15, 20, 25, 30 or greater than 30 days. Preferably the plant or part thereof, such as a seed, treated with (e.g. coated with) the bacteria is substantially stable at temperatures between about 4° C. and about 37° C. for at least one year or greater than one year. In certain embodiments, the plant or part thereof treated with (e.g. coated with) the bacteria are confined within a suitable container, such as an object selected from the group consisting of: bottle, jar, ampule, package, vessel, bag, box, bin, envelope, carton, container, silo, shipping container, truck bed, and case.

In certain embodiments, the bacteria as taught herein are heterologous to the plant or plant part to be treated or administered with the same. A bacterium is considered heterologous to the plant, plant part, seed for growing the plant, or locus of the plant if the plant, plant part, seed for growing the plant, or locus of the plant that is untreated (e.g., a seed that is not treated with a bacterial strain described herein) does not contain detectable levels of the bacterium. A bacterium is considered “heterologously disposed” or “heterologous disposed” on the exterior surface of or within a plant or plant tissue when the bacterium is applied or disposed on the plant in a number that is not found on that plant before application of the bacterium. For example, a purified bacterial strain disposed on an exterior surface or within the seed can be an endophytic bacterium that may be associated with the mature plant, but is not found on the surface of or within the seed. As such, a bacterium is deemed heterologously disposed when applied on the plant that either does not naturally have the bacterium on its surface or within the particular tissue to which the bacterium is disposed, or does not naturally have the bacterium on its surface or within the particular tissue in the number that is being applied.

In certain embodiments, the strain is heterologous disposed, for example, on the surface of a reproductive element of a plant, in an amount effective to be detectable in the mature plant. In a particular embodiment, the strain is heterologous disposed in an amount effective to be detectable in an amount of at least about 100 CFU, between 100 and 200 CFU, at least about 200 CFU, between 200 and 300 CFU, at least about 300 CFU, between 300 and 400 CFU, at least about 500 CFU, between 500 and 1,000 CFU, at least about 1,000 CFU, between 1,000 and 3,000 CFU, at least about 3,000 CFU, between 3,000 and 10,000 CFU, at least about 10,000 CFU, between 10,000 and 30,000 CFU, at least about 30,000 CFU, between 30,000 and 100,000 CFU, at least about 100,000 CFU or more in the mature plant.

In a preferred embodiment, the bacteria are heterologously disposed to a plant, part thereof, seed for growing the plant, or locus of the plant in an amount effective to confer a pathogen control or resistance, such as a reduced disease severity or necrosis relative to an untreated plant. In a preferred embodiment, the amount of the heterologous disposed strain to the plant, part thereof, seed for growing the plant, or locus of the plant is effective to maintain a healthy state in the plant. In a further embodiment, the amount of the heterologous disposed strain to a seed for growing a plant is effective to maintain a healthy state in the mature plant germinated from the seed. Hence, in certain embodiments, any of the bacterial strains, bacterial populations, or microbial active ingredients as contemplated herein may be heterologous disposed to the plant, plant part, or seed. The present application also provides aspects and embodiments as set forth in the following Statements. In these statements, the wording “The [subject] according to Statement [number], wherein . . . ” or “The [subject] according to any one of Statements [numbers], wherein . . . ” also discloses and may be replaced by the simple wording “In certain embodiments . . . ”.

Statement 1. A method for conferring a pathogen control or resistance in a plant, the method comprising administering bacteria of a bacterial strain which comprises a 16S polynucleotide having 100% sequence identity to SEQ ID NO: 1 to the plant, a part thereof, a seed for growing the plant, or a locus of the plant.

Statement 2. A method for conferring a pathogen control or resistance in a plant, the method comprising administering a microbial active ingredient that is derived from a bacterial strain which comprises a 16S polynucleotide having 100% sequence identity to SEQ ID NO: 1 to the plant, a part thereof, a seed for growing the plant, or a locus of the plant.

Statement 3. The method according to Statement 1 or 2, wherein:
the pathogen is a bacterium or fungus;
the pathogen is a fungus;
the pathogen is selected from the group consisting of Fusarium spp., Phytophthora spp., Pythium spp., Rhizoctonia spp., Puccinia spp., Mycosphaerella spp., Sclerotinia spp., Botrytis spp., Colletotrichum spp., Microdochium spp., Gaeumannomyces spp., Tapesia spp., Ustilago spp., Ramularia spp., Blumeria spp., Podosphaera spp., Alternaria spp., Monilinia spp., and Penicillium spp; or
the pathogen is Fusarium spp.

Statement 4. The method according to any one of the Statements 1 to 3, wherein the bacterial strain comprises a 16S polynucleotide as set forth in SEQ ID NO: 1.

Statement 5. The method according to any one of Statements 1 to 4, wherein the bacterial strain is a Streptomyces bottropensis strain.

Statement 6. The method according to any one of Statements 1 to 5, wherein the bacterial strain is selected from the group consisting of:
the strain deposited under the Budapest Treaty at the Polish Collection of Microorganisms (PCM) on 11 Feb. 2022 under Accession No. B/00366, or a functional mutant thereof;
the strain deposited under the Budapest Treaty at the Polish Collection of Microorganisms (PCM) on 26 Nov. 2021 under Accession No. B/00364, or a functional mutant thereof;
the strain deposited under the Budapest Treaty at the Polish Collection of Microorganisms (PCM) on 2 Nov. 2022 under Accession No. B/00435, or a functional mutant thereof;
the strain deposited under the Budapest Treaty at the Polish Collection of Microorganisms (PCM) on 2 Nov. 2022 under Accession No. B/00436, or a functional mutant thereof;
the strain deposited under the Budapest Treaty at the Polish Collection of Microorganisms (PCM) on 2 Nov. 2022 under Accession No. B/00437, or a functional mutant thereof;
the strain deposited under the Budapest Treaty at the Polish Collection of Microorganisms (PCM) on 2 Nov. 2022 under Accession No. B/00438, or a functional mutant thereof; and
combinations thereof;

Statement 7. The method according to any one of Statements 1 to 5, wherein the bacterial strain is selected from the group consisting of:
the strain deposited under the Budapest Treaty at the Polish Collection of Microorganisms (PCM) on 11 Feb. 2022 under Accession No. B/00366;
the strain deposited under the Budapest Treaty at the Polish Collection of Microorganisms (PCM) on 26 Nov. 2021 under Accession No. B/00364;
the strain deposited under the Budapest Treaty at the Polish Collection of Microorganisms (PCM) on 2 Nov. 2022 under Accession No. B/00435;
the strain deposited under the Budapest Treaty at the Polish Collection of Microorganisms (PCM) on 2 Nov. 2022 under Accession No. B/00436;
the strain deposited under the Budapest Treaty at the Polish Collection of Microorganisms (PCM) on 2 Nov. 2022 under Accession No. B/00437;
the strain deposited under the Budapest Treaty at the Polish Collection of Microorganisms (PCM) on 2 Nov. 2022 under Accession No. B/00438; and
combinations thereof.

Statement 8. The method according to any one of Statements 1 to 7, wherein the bacterial strain is B/00366.

Statement 9. The method according to any one of Statements 1 to 8, comprising administering to the plant, the part thereof, the seed for growing the plant, or the locus of the plant:
bacteria of two or more bacterial strains, each independently as defined in any one of Statements 1-6, and/or
the bacteria in conjunction with one or more additional plant-beneficial microorganism.

Statement 10. The method according to any one of Statements 2 to 9, comprising administering to the plant, the part thereof, the seed for growing the plant, or the locus of the plant:
a microbial active ingredient that is derived from two or more bacterial strains, each independently as defined in any one of Statements 1 to 7, and/or
the microbial active ingredient in conjunction with one or more additional plant-beneficial microorganism,
   wherein the microbial active ingredient is a supernatant from a culture of the bacterial strain or an extract or extract fraction from a culture of the bacterial strain.

Statement 11. The method according to any one of Statements 1 to 10, wherein the bacteria or the microbial active ingredient are administered in an agricultural active composition, such as wherein:
the composition further comprises one or more agriculturally acceptable auxiliary, such as a solvent, a carrier, a surfactant, a sticker, an antifreeze agent, a thickener, a buffering agent, an antifoaming agent, an antioxidant, a preservative, an aroma, a colorant, or a combination thereof;
the composition is a liquid composition, such as an aqueous composition, such as a sprayable liquid or a concentrate, preferably wherein the composition comprises the bacteria at a concentration of at least about 10² CFU/ml; or
the composition is a non-liquid composition, such as a powder, preferably wherein the composition comprises the bacteria at an amount of at least about 10² CFU/g.

Statement 12. A bacterial strain selected from the group consisting of:
the strain deposited under the Budapest Treaty at the Polish Collection of Microorganisms (PCM) on 11 Feb. 2022 under Accession No. B/00366, or a functional mutant thereof;
the strain deposited under the Budapest Treaty at the Polish Collection of Microorganisms (PCM) on 26 Nov. 2021 under Accession No. B/00364, or a functional mutant thereof;
the strain deposited under the Budapest Treaty at the Polish Collection of Microorganisms (PCM) on 2 Nov. 2022 under Accession No. B/00435, or a functional mutant thereof;
the strain deposited under the Budapest Treaty at the Polish Collection of Microorganisms (PCM) on 2 Nov. 2022 under Accession No. B/00436, or a functional mutant thereof;
the strain deposited under the Budapest Treaty at the Polish Collection of Microorganisms (PCM) on 2 Nov. 2022 under Accession No. B/00437, or a functional mutant thereof; and
the strain deposited under the Budapest Treaty at the Polish Collection of Microorganisms (PCM) on 2 Nov. 2022 under Accession No. B/00438, or a functional mutant thereof; or
   a combination of two or more of said strains; or a bacterial population comprising one or more of said strains.

Statement 13. A bacterial strain selected from the group consisting of:
the strain deposited under the Budapest Treaty at the Polish Collection of Microorganisms (PCM) on 11 Feb. 2022 under Accession No. B/00366;
the strain deposited under the Budapest Treaty at the Polish Collection of Microorganisms (PCM) on 26 Nov. 2021 under Accession No. B/00364f;
the strain deposited under the Budapest Treaty at the Polish Collection of Microorganisms (PCM) on 2 Nov. 2022 under Accession No. B/00435;
the strain deposited under the Budapest Treaty at the Polish Collection of Microorganisms (PCM) on 2 Nov. 2022 under Accession No. B/00436;
the strain deposited under the Budapest Treaty at the Polish Collection of Microorganisms (PCM) on 2 Nov. 2022 under Accession No. B/00437; and
the strain deposited under the Budapest Treaty at the Polish Collection of Microorganisms (PCM) on 2 Nov. 2022 under Accession No. B/00438; or
   a combination of two or more of said strains; or a bacterial population comprising one or more of said strains.

Statement 14. An agricultural active composition comprising one or more of the bacterial strains as defined in any one of Statements 1 to 7, 12 or 13.

Statement 15. The agricultural active composition of Statement 14, further comprising one or more agriculturally acceptable auxiliary, such as a solvent, a carrier, a surfactant, a sticker, an antifreeze agent, a thickener, a buffering agent, an antifoaming agent, an antioxidant, a preservative, an aroma, a colorant, or a combination thereof.

Statement 16. The agricultural active composition of Statement 14 or 13, wherein
the composition is a liquid composition, such as an aqueous composition, such as a sprayable liquid or a concentrate, preferably wherein the composition comprises the bacteria at a concentration of at least about 10² CFU/ml; or
the composition is a non-liquid composition, such as a powder, preferably wherein the composition comprises the bacteria at an amount of at least about 10² CFU/g.

Statement 17. The agricultural active composition of any one of Statements 14 to 16 wherein the bacterial strain is present in spray-dried or freeze-dried form.

Statement 18. A microbial active ingredient capable of conferring a pathogen control or resistance in a plant, characterized in that the ingredient is derived from the bacterial strain as defined in any of Statements 1 to 7, 12 or 13.

Statement 19. A plant or plant part heterologously disposed with bacteria of the bacterial strain as defined in any one of Statements 1 to 7, 12 or 13 or coated with the agricultural active composition according to any of the Statements 14 to 16.

The above aspects and embodiments are further supported by the following non-limiting examples.

### EXAMPLES

### Example 1: Storage, Cultivation, and Formulation of Bacteria According to Certain Embodiments of the Invention

The bacterial strain deposited the Polish Collection of Microorganisms (PCM) on 11 Feb. 2022 under Accession No. B/00366 (with the following identification reference given to the deposited material by the depositor: MED-B-M14B4) (proposed taxonomic designation Streptomcyes bottropensis) (henceforth referred to in the Examples as “the M14B4 strain”), the bacterial strain deposited at the Polish Collection of Microorganisms (PCM) on 26 Nov. 2021 under Accession No. B/00364 (with the following identification reference given to the deposited material by the depositor: MED-B-M2F4) (proposed taxonomic designation Streptomyces bottropensis) (henceforth referred to in the Examples as “the M2F4 strain”), the bacterial strain deposited at the Polish Collection of Microorganisms (PCM) on 2 Nov. 2022 under Accession No. B/00435 (with the following identification reference given to the deposited material by the depositor: MED-B-M14B2) (proposed taxonomic designation Streptomyces bottropensis) (henceforth referred to in the Examples as “the M14B2 strain”), the bacterial strain deposited at the Polish Collection of Microorganisms (PCM) on 2 Nov. 2022 under Accession No. B/00436 (with the following identification reference given to the deposited material by the depositor: MED-B-M14B3) (proposed taxonomic designation Streptomyces bottropensis) (henceforth referred to in the Examples as “the M14B3 strain”), the bacterial strain deposited at the Polish Collection of Microorganisms (PCM) on 2 Nov. 2022 under Accession No. B/0047 (with the following identification reference given to the deposited material by the depositor: MED-B-M14B1) (proposed taxonomic designation Streptomyces bottropensis) (henceforth referred to in the Examples as “the M14B1 strain”); the bacterial strain deposited at the Polish Collection of Microorganisms (PCM) on 2 Nov. 2022 under Accession No. B/00438 (with the following identification reference given to the deposited material by the depositor: MED-B-M2H3) (proposed taxonomic designation Streptomyces bottropensis) (henceforth referred to in the Examples as “the M2H3 strain”) were stored at −75° C. in nutrient broth (beef extract 0.3% w/v, peptone 0.5% w/v, 0.5% w/v sodium chloride, in distilled water, pH 6.8±0.2) amended with 20% v/v glycerol and was cultured on nutrient agar (composition as nutrient broth with 1.5% w/v agar) for at least three days at 28° C. NA medium is also available from commercial sources, such as Merck KGaA/Sigma-Aldrich (Darmstadt, Germany) product number 70149. Cell suspensions were prepared by harvesting cells which have been incubated from single colonies in nutrient broth between two and five days in a shaker at 28° C. degrees. Harvesting was done through centrifugation at 5000 g for 5 minutes. Alternatively, the whole cell broth, comprising the bacteria as well as the medium in which the bacteria can be used for inoculation.

For in planta testing of the effect of the bacterial strains on fungal pathogens in climate-controlled growth chambers, living biomass of the bacterial strains of the invention was produced in NB, centrifuged to concentrate the cells, resuspended in an appropriate volume of sterile water with Tween20 for foliar application of spikes of wheat plants by means of dipping or spray application. One day after the application of the biomass of the strains of the invention acting as biocontrol agent (BCA) to the wheat spikes, also the fungal plant pathogen was applied by means of dipping or spray application.

Alternatively, for applying spores of the bacterial strains of the invention instead of living biomass, said spores were produced using solid-state fermentation techniques on an organic carrier, soaked in water with nutrients and additives to enhance sporulation. The spores were separated from the carrier by washing them off in water or water with compounds that serve as a pH buffer or dispersant. The spores were stored at −75° C. by supplementing the harvested liquid with 20 v/v % glycerol. The spores were subsequently applied to the wheat spikes using the same preparation procedure as for liquid produced living biomass as described above. Also in this case, the fungal plant pathogen was subsequently applied by means of dipping or spray application.

The effectivity of bacterial strain as a bio-control (in particular, bio-fungicide) agent was in both cases determined as set forth in the Examples below, such as by the level of necrosis of the plant as compared to an untreated control, or by means of image analysis.

### Example 2: In Vitro Co-Culturing Experiments with Fungal Phytopathogens and Bacterial Strains

An in vitro co-culturing experiment is used to determine the growth inhibition effect of bacterial strains B/00366, B/00364, M14B1, M14B2, M14B3 and M2H3 on different phytopathogenic fungi, namely Alternaria solani CABI 46816, Fusarium oxysporum MUCL 39793, Microdochium nivale MUCL 18523, Monilinia fructigena CABI 223846, Penicillium digitatum CABI 91956, Pythium sulcatum CBS 603.73, Rhizoctonia solani MUCL 18834, Sclerotinia sclerotiorum MUCL 9083, different strains of Botrytis cinerea (CABI 169558, CABI 169559, CBS 810.69 and MUCL 399) and Fusarium graminearum (BRFM 1995, BRFM 1982 and BRFM 1977).

These fungi were available as public deposits under the respective accession numbers mentioned above from: CABI—CABI, Bakeham Lane, Englefield Green, Egham, Surrey TW20 9TY, United Kingdom; MUCL—BCCM™//MUCL Agro-industrial fungi, yeasts and arbuscular mycorrhizal fungi collection, Université catholique de Louvain, Mycothèque de l'Université catholique de Louvain, Croix du Sud, 3—box L7.05.06, 1348 Louvain-La-Neuve, Belgium; CBS—Westerdijk Fungal Biodiversity Institute (CBS), Uppsalalaan 8, 3584 CT Utrecht, The Netherlands; BRFM: CIRM-CF/FILAMENTOUS FUNGI, UMR 1163 Biodiversité et Biotechnologie Fongiques, Polytech-Génie Biologique, 163 av de Luminy, CP 925, 13288 Marseille Cedex 09, France.

A co-culturing experiment of a bacterial strain member of the species Streptomyces bottropensis B/00366, B/00364, M14B1, M14B2, M14B3 and M2H3 is executed with phytopathogenic fungi. Potato Dextrose Agar (PDA) medium is prepared and dispensed over petri dishes with a diameter of 8.5 cm. Each purified bacterial strain is cultured in a liquid Nutrient broth until a dense bacterial culture is obtained. An amount, in particular 15 μl, of the liquid culture is taken and inoculated at 2.5 cm from the center of the petri dish. The petri dish is incubated at 28° C. overnight. Thereafter, the center of the petri dish is inoculated with 15 μl of the phytopathogenic fungi grown in Potato Dextrose Broth (PDB) and incubated at 21° C. and 70% RH. Control petri dishes inoculated only with the phytopathogenic fungi are prepared as specified above to follow the fungal growth and assessment date. The assay is scored when the inoculated phytopathogenic fungi has reached the edge of the control petri dishes. The growth inhibition zone is scored as A, B, C, D or E depending on the fungal growth, wherein score A is given when fungal growth is limited to 1 cm from the fungal inoculation point, score B is given when fungal growth is observed up to 2 cm from the fungal inoculation point A, score C is given when the fungal pathogen reaches the bacterial growth line, score D is given when the fungal pathogen reaches the bacterial growth line but not the edge of the petri dish and score E is considered as no effect, being the fungal pathogen able to outcompete the bacterial growth and reach the edges of the petri dish (Table 1). These co-culturing experiments underline the broad-spectrum disease control in plants by the Streptomyces bottropensis strains according to the embodiments of the current invention. Said strains clearly inhibit the growth of several fungal pathogens as listed in Table 1.

TABLE 1

| Fungal pathogen (strain) | B/00366 | B/00364 | M14B1 | M14B2 | M14B3 | M2H3 |
|---|---|---|---|---|---|---|
| Alternaria solani | E | D | C | C | C | B |
| (CABI 46816) | | | | | | |
| Botrytis cinerea | B | B | B | B | B | B |
| (MUCL 399) | | | | | | |
| Botrytis cinerea | B | E | B | B | B | B |
| (CABI 169558) | | | | | | |
| Botrytis cinerea | B | B | B | B | B | B |
| (CABI 169559) | | | | | | |
| Botrytis cinerea | B | B | B | B | B | B |
| (CBS 810.69) | | | | | | |
| Fusarium graminearum | B | B | B | B | B | B |
| (BRFM 1982) | | | | | | |
| Fusarium graminearum | E | A | B | B | A | E |
| (BRFM 1977) | | | | | | |
| Microdochium nivale | A | A | B | A | A | A |
| (MUCL 18523) | | | | | | |
| Monilinia fructigena | C | B | NA | B | B | A |
| (CABI 223846) | | | | | | |
| Penicilium digitatum | B | B | B | B | B | B |
| (CABI 91956) | | | | | | |
| Pythium sulcatum | B | E | C | E | E | B |
| (CBS 603.73) | | | | | | |
| Rhizoctonia solani | B | E | E | D | D | D |
| (MUCL 18834) | | | | | | |
| Sclerotinia sclerotiorum | B | B | B | B | B | B |
| (MUCL 9083) | | | | | | |

### Example 3: In Planta Inhibition of Fungal Pathogen Fusarium graminearum

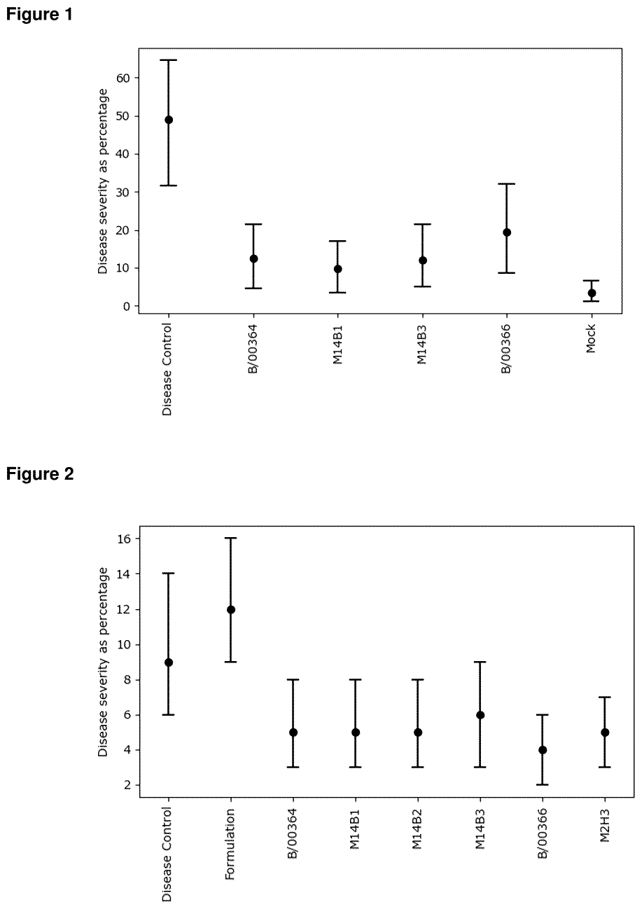

Fusarium graminearum (Fg), also referred to as Gibberella zeae, is a fungal plant pathogen causing Fusarium Head Blight (FHB) on wheat and barley. Bacterial strains B/00364, M14B1, M14B3 and B/00366 were used for in planta inhibition experiments of the fungus on wheat (Triticum aestivum). For the bacterial treatments, flowering spikes of adult wheat plants were treated preventively with the formulated bacterial strains (bacteria were formulated in 0.5% carboxymethyl cellulose (CMC) in PBS buffer, at concentration of 10⁶ CFU/ml). Two or three days later, the plants were treated with formulated spores of Fg (spores were formulated in 0.18% TWEEN20 in sterile water, at concentration of 10⁴-10⁵ spores/ml). The controls were a disease control and a formulation control with no pathogen applied later (Mock). After 2 to 3 weeks in a growth chamber, disease severity (%), in particular % of necrosis caused by the Fusarium pathogen, was measured using Fusarium Vision, an in-house developed algorithm linked to an imaging technology for Fusarium disease scoring on wheat spikes. Results are shown in FIG. 1.

Wheat spikes treated with the strains B/00364, M14B1, M14B3 and B/00366 showed a decrease in disease severity of −69%, −75%, −71% and −56% respectively, compared to the disease control.

### Example 4: In Planta Inhibition of Fungal Pathogen Puccinia striiformis var. tritici

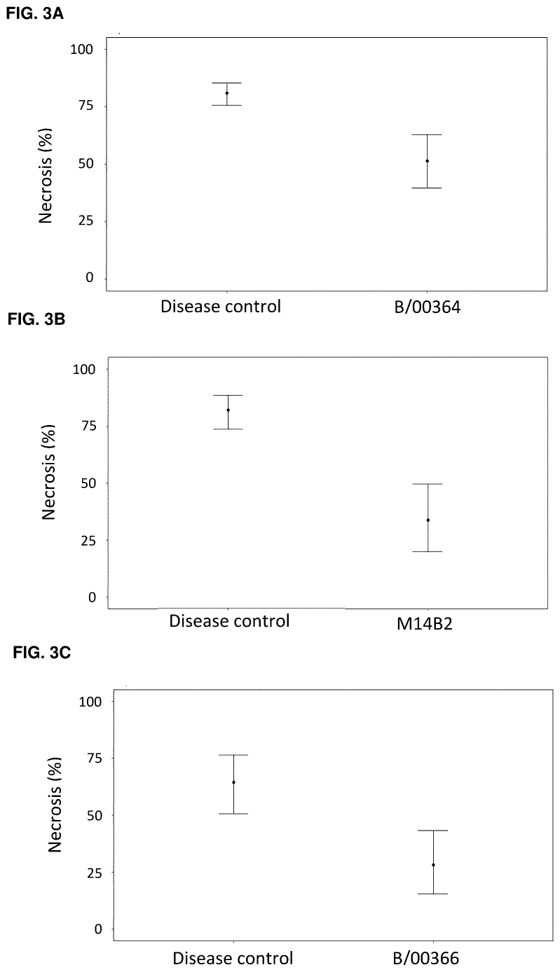

Puccinia striiformis var. tritici is a plant pathogen causing yellow rust (also called stripe rust) on wheat. In analogy to Example 3, bacterial strains B/00364, M14B1, M14B2, M14B3, B/00366 and M2H3 were used for in planta inhibition experiments of the fungus P. striiformis var. tritici on wheat leaves. Single leaves were treated with the formulated bacterial strains (bacteria were formulated in 0.01% polyvinyl alcohol (PVA), 0.02% TWEEN20 in sterile water, at concentration of 10⁶ CFU/ml) as a preventive treatment and after a short drying period, the plants were treated with P. striiformis var. tritici spores (spores were formulated in Novec®, 3M, 3 mg/ml). As a disease control, single leaves were sprayed with P. striiformis var. tritici spores only. In a formulation control (Formulation), single leaves were applied with only the formulation in which the bacteria were formulated (but without the bacteria), and after a short drying period, the plants were treated with P. striiformis var. tritici spores. After 2 to 3 weeks in a growth chamber, disease pressure (%) was measured using Puccinia Vision, an in-house developed algorithm linked to an imaging technology for Puccinia disease scoring on wheat leaves. The algorithm measures the disease severity by defining the % diseased leaf area. Results are shown in FIG. 2.

The wheat leaves treated with the strains B/00364, M14B1, M14B2, M14B3, B/00366 and M2H3 showed a decrease in disease severity of −48%, −46%, −33%, −26%, −43% and −41% respectively, compared to the disease control.

### Example 5: In Planta Inhibition of Fungal Pathogen Zymoseptoria tritici

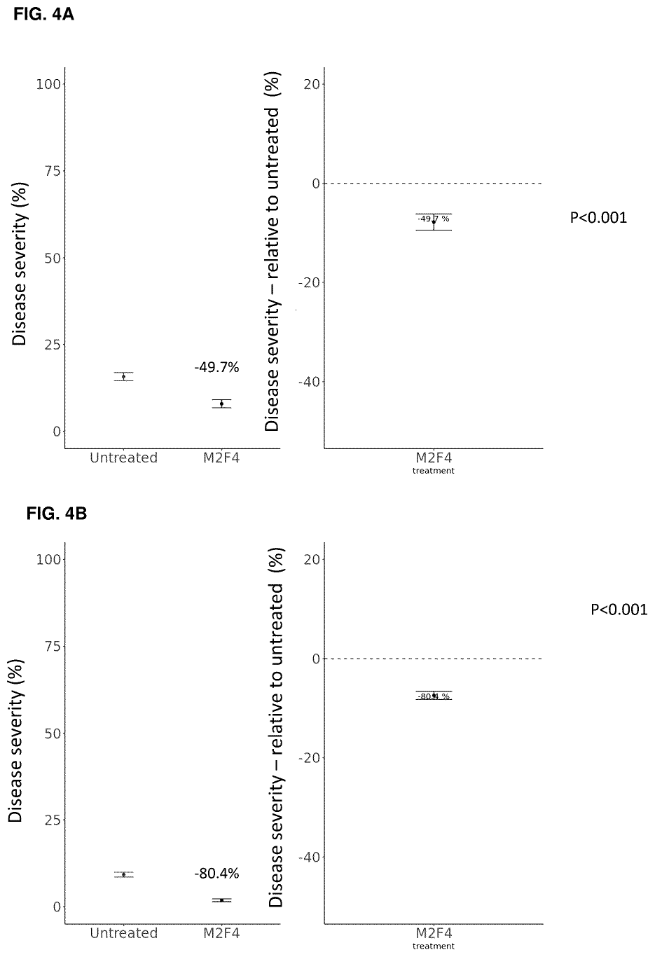

Zymoseptoria tritici, also referred to as Mycosphaerella graminicola or Septoria tritici, is a fungal wheat plant pathogen causing Septoria Leaf Blotch. In analogy to Example 3 and 4, bacterial strains B/00364, M14B2 and B/00366 were used for in planta inhibition experiments of the fungus Z. tritici on wheat leaves. For the bacterial treatment, wheat leaves were treated preventively with the bacterial strain (bacteria were formulated in 0.18% TWEEN20 and 0.01% PVA in sterile water, at concentration of 10⁶ CFU/ml). Two to three days later, the leaves were treated with Z. tritici spores (spores were formulated in 0.18% TWEEN20 in sterile water, at concentration of 10⁶ spores/ml). As a disease control, wheat leaves were treated with Z. tritici spores only. Disease severity (as % necrosis) was measured after 3 to 4 weeks in a growth chamber using visual assessment by trained experimenters. Results are shown in FIGS. 3A-C.

The wheat leaves treated with the strains B/00364, M14B2 and B/00366 showed a decrease in disease severity −37%, −60% and −56% respectively, compared to the disease control.

### Example 6: Reduction of the Fungal Disease Fusarium Head Blight Severity Caused by Fusarium sp. In Triticum sp. In Field Condition

To evaluate the efficacy of biocontrol agents (bacterial strains of the embodiments of the invention) for Fusarium Head Blight (F. graminearum) in wheat, the Findus wheat variety was tested in 24 m² plots in Gietrzwałd, Poland. Treatments included two untreated, two mock treatments (formulation without microorganisms), one commercial preventive fungicide treatment (Aviator® Xpro, Bayer Crop Science), one commercial biocontrol reference treatment (Echiquier®, De Sangosse S.A.S.) and 9 biocontrol treatments which include one using the bacterial strain M2F4 (a water dispersible granule (WG) sprayed at 4.00E+11 CFU/ha) according to certain embodiments of the present invention. A properly randomized design was used with 8 replicates for untreated treatment and four replicates for all other treatments.

Artificial inoculation with F. graminearum spores was done the evening after the second application by spraying the Fusarium spore solution (at a concentration of 10⁵ spores per ml solution) full field on the trial with a spraying boom (500 l water/ha, 3 bar). In the next 48 hours irrigation was performed.

Biocontrol inoculation was performed in two phases, first at BBCH 61 (10% anthesis), equivalent to the beginning of flowering and the second 2 days later at BBCH63-65, equivalent to 30-50% anthesis. To assess phytotoxicity, a visual estimation was done 5-7 days after the first application of the treatments. Disease severity was assessed by % of the spike affected by Fusarium, assessed on 50 spikes/plot, as recommended at EPPO guidelines PP1/026 (4)—Foliar and ear diseases on cereals. Disease severity was evaluated twice: the first time at +−15% average disease severity in the untreated and the second time at +−40% average disease severity in the untreated. All the assessments were performed as recommended in the EPPO guidelines PP1/026 (4).

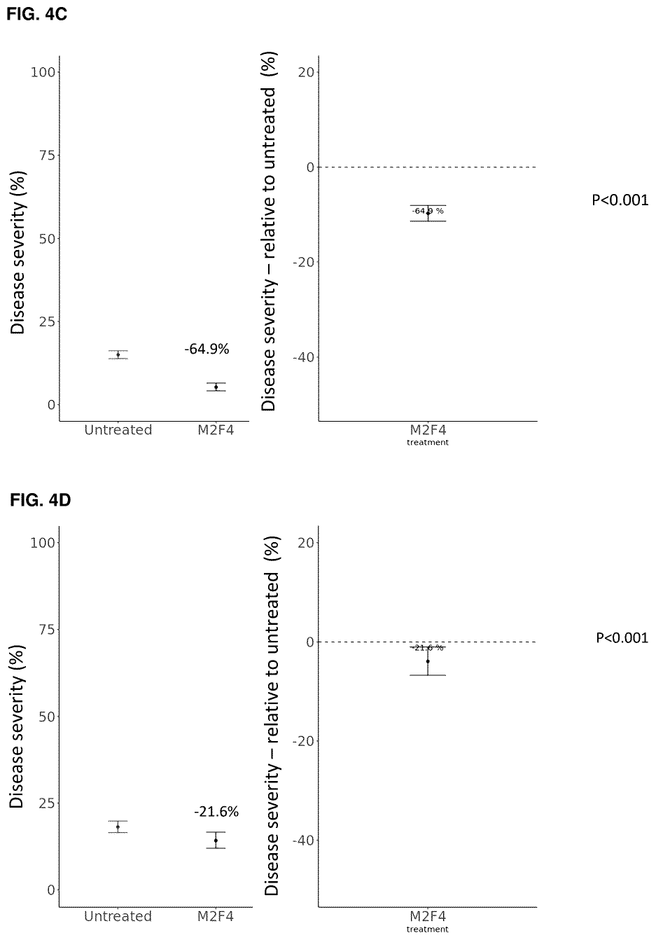

The untreated treatment showed a disease severity of 15.69%. In the treatment where M2F4 was applied, a disease severity of 7.89% was observed, which was significantly lower (P=0.00—a reduction of 49.7%) than the untreated treatment (FIG. 4A). The chemical fungicide Aviator® Xpro and the commercial biocontrol reference treatment (Echiquier®) showed a disease severity of 3.91% and 6.01% respectively (not shown).

A similar experiment to evaluate the efficacy of the bacterial strains of the invention for Fusarium Head Blight (F. graminearum) in wheat was repeated on fields in Bulgaria following exactly the same protocol as described above. Results are shown in FIG. 4B. Also here, a significant reduction of disease severity (−80%) was seen in plants treated with M2F4 compared to untreated control.

### Example 7: Reduction of the Fungal Disease Caused by Blumeria Graminis f. Sp. Tritici in Triticum Sp. In Field Condition

To evaluate the efficacy of biocontrol agents against powdery mildew (B. graminis) in wheat, a field trial was set-up in France. Treatments included three untreated, two mock treatments (formulation without microorganisms), one commercial preventive fungicide treatment (Nissodium, Certis), one commercial biocontrol reference treatment (Heliosoufre S®, Helioterpen) and 8 biocontrol treatments including one using the bacterial strain M2F4 (a water dispersible granule (WG) sprayed at 2.00E+13 CFU/ha) according to certain embodiments of the present invention. A properly randomized design was used with 12 replicates for untreated treatment and four replicates for all other treatments.

Biocontrol inoculation was performed twice. First, an application was performed when the first disease symptoms were visible on the lower leaf ranks (L4-L5 counting from the bottom). Then, a second application was performed 5 days later. A natural infestation of powdery mildew occurred, no artificial inoculation was performed in this trial.

To assess phytotoxicity, a visual estimation was done 5-7 days after the first application of the treatments. Disease severity was assessed by % of the leaf affected by powdery mildew, assessed on 25 leaves/plot, as recommended at EPPO guidelines PP1/026(4)—Foliar and ear diseases on cereals. Disease severity was evaluated three times: the first time at ≈15% average disease severity in the untreated, the second time at ≈25% average disease severity in the untreated and the third time at ≈50% average disease severity on the untreated. All the assessments were performed as recommended in the EPPO guidelines PP1/026 (4).

In the treatment where M2F4 was applied, a significant reduction of disease severity was observed (p-val.=0.00), with an observed relative disease reduction of 64.9% (FIG. 4C).

Similar experiments using B. graminis were performed on a location in France; results are shown in FIG. 4D. A relative reduction of disease severity was observed seen in plants treated with M2F4 compared to untreated controls: −21.6% France (p<0.001).

### Example 8: Reduction of Powdery Mildew Caused by Fungal Pathogen Podosphaera xanthii Greenhouse Conditions

To evaluate the efficacy of biocontrol agents, the bacterial strains of the embodiments of the invention, against powdery mildew in cucurbits a trial was set up in a greenhouse in Italy in cucumber.

Treatments included two untreated, one mock treatment (formulation without microorganisms), one commercial chemical fungicide treatment (Flint® Max, Bayer), one biological reference (Sonata®, Bayer) treatment and 5 biocontrol treatments which included the bacterial strain M2F4 (a water dispersible granule (WG) sprayed at 7,50E+12 CFU/ha) according to certain embodiments of the present invention. The first application was applied preventively, the following 7 applications were applied with an interval of 5 to 7 days.

The efficacy assessments on the leaves (upper and lower leaf surface) were conducted at the disease appearance in all plots. The following assessments were carried out before each application and the last assessment 10 days after the last application according to the EPPO guidelines PP1/57 (3)-Powdery mildews on cucurbits and other vegetables.

The % of leaf area affected on both upper and lower surfaces of at least four leaves of uniform age was assessed on at least eight plants per plots.

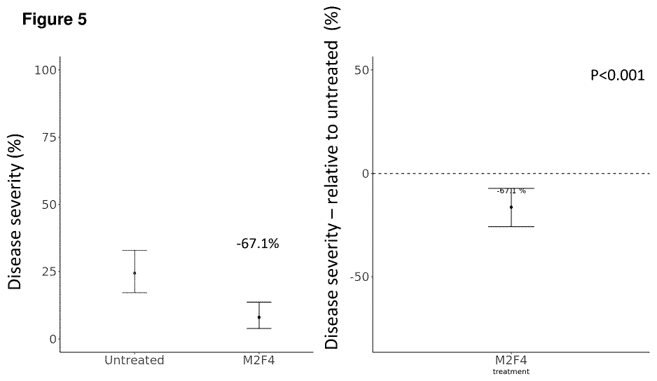

The untreated treatment showed a disease severity of 24.39%. The treatment where M2F4 was applied showed a disease severity of 8.02%, which was significantly lower (P<0.001—a reduction of 67.1%) than the untreated treatment. Results are shown in FIG. 5. The chemical fungicide Flint® Max showed disease severity of 2.77% (not shown) whereas for the biological reference treatment a disease severity of 9.78% was observed (not shown).

### DEPOSIT OF BIOLOGICAL MATERIAL

The purified bacterial strains as taught herein are deposited under the terms of the Budapest Treaty, as follows:
Strain MED-B-M14B4 deposited under Accession No. B/00366;
Strain MED-B-M2F4 deposited under Accession No. B/00364;
Strain MED-B-M14B2 deposited under Accession No. B/00435;
Strain MED-B-M14B3 deposited under Accession No. B/00436;
Strain MED-B-M14B1 deposited under Accession No. B/00437;
Strain MED-B-M2H3 deposited under Accession No. B/00438.

Table 2 summarizes the requisite indications relating to these deposited microorganisms referred to throughout this specification.

TABLE 2

| Accession | | | | | | |
|---|---|---|---|---|---|---|
| number given | | | | | | |
| by depositary | | | | | | |
| institution | B/00366 | B/00364 | B/00435 | B/00436 | B/00437 | B/00438 |
| Identification | MED-B- | MED-B- | MED-B- | MED-B- | MED-B- | MED-B- |
| reference | M14B4 | M2F4 | M14B2 | M14B3 | M14B1 | M2H3 |
| given by the | | | | | | |
| depositor | | | | | | |
| Proposed | Streptomyces bottropensis | | | | | |
| taxonomic | | | | | | |
| designation | | | | | | |
| Date of | 11 Feb. | 26 Nov. | 2 Nov. | 2 Nov. | 2 Nov. | 2 Nov. |
| deposit | 2022 | 2021 | 2022 | 2022 | 2022 | 2022 |
| Depositary | Polish Collection of Microorganisms (PCM) | | | | | |
| institution, | Institute of Immunology and Experimental Therapy | | | | | |
| and address | Polish Academy of Sciences | | | | | |
| | UI. Weigla 12 | | | | | |
| | 53-114 Wroclaw | | | | | |
| | Poland | | | | | |
| Depositor | Aphea. Bio NV | | | | | |
| Address of | Technologiepark 21, B-9052 Zwijnaarde, Belgium | | | | | |
| depositor | | | | | | |

### SEQUENCE LISTING

Throughout the description and examples, reference is made to the following sequences, as shown in Table 3:
SEQ ID NO: 1: Nucleotide sequence of 16S polynucleotide from strains B/00366, B/00364, B/00435, B/00436, B/00437, and B/00438.

TABLE 3

| Strain | SEQ ID NO | Sequence of 16S polynucleotide (5′-3′) |
|---|---|---|
| B/00366 | 1 | ACGGAGAGTTTGATCCTGGCTCAGGACGAACGCTGGCGGCGTGCTTAACA |
| B/00364 | | CATGCAAGTCGAACGATGAACCACTTCGGTGGGGATTAGTGGCGAACGGG |
| B/00435 | | TGAGTAACACGTGGGCAATCTGCCCTGCACTCTGGGACAAGCCCTGGAAA |
| B/00436 | | CGGGGTCTAATACCGGATAACACACGCTCAGGCATCTGAGTGTGTTGAAA |
| B/00437 | | GCTCCGGCGGTGCAGGATGAGCCCGCGGCCTATCAGCTTGTTGGTGAGGT |
| B/00438 | | AGAAGCTCACCAAGGCGACGACGGGTAGCCGGCCTGAGAGGGCGACCGG |
| | | CCACACTGGGACTGAGACACGGCCCAGACTCCTACGGGAGGCAGCAGTGG |
| | | GGAATATTGCACAATGGGCGAAAGCCTGATGCAGCGACGCCGCGTGAGG |
| | | GATGACGGCCTTCGGGTTGTAAACCTCTTTCAGCAGGGAAGAAGCGAAAG |
| | | TGACGGTACCTGCAGAAGAAGCGCCGGCTAACTACGTGCCAGCAGCCGCG |
| | | GTAATACGTAGGGCGCAAGCGTTGTCCGGAATTATTGGGCGTAAAGAGCT |
| | | CGTAGGCGGCTTGTCACGTCGGTTGTGAAAGCCCGGGGCTTAACCCCGGG |
| | | TCTGCAGTCGATACGGGCAGGCTAGAGTGTGGTAGGGGAGATCGGAATTC |
| | | CTGGTGTAGCGGTGAAATGCGCAGATATCAGGAGGAACACCGGTGGCGA |
| | | AGGCGGATCTCTGGGCCATTACTGACGCTGAGGAGCGAAAGCGTGGGGA |
| | | GCGAACAGGATTAGATACCCTGGTAGTCCACGCCGTAAACGGTGGGAACT |
| | | AGGTGTTGGCGACATTCCACGTCGTCGGTGCCGCAGCTAACGCATTAAGTT |
| | | CCCCGCCTGGGGAGTACGGCCGCAAGGCTAAAACTCAAAGGAATTGACGG |
| | | GGGCCCGCACAAGCAGCGGAGCATGTGGCTTAATTCGACGCAACGCGAAG |
| | | AACCTTACCAAGGCTTGACATATACCGGAAAGCATCAGAGATGGTGCCCCC |
| | | CTTGTGGTCGGTATACAGGTGGTGCATGGCTGTCGTCAGCTCGTGTCGTGA |
| | | GATGTTGGGTTAAGTCCCGCAACGAGCGCAACCCTTGTTCTGTGTTGCCAG |
| | | CATGCCCTTCGGGGTGATGGGGACTCACAGGAGACTGCCGGGGTCAACTC |
| | | GGAGGAAGGTGGGGACGACGTCAAGTCATCATGCCCCTTATGTCTTGGGC |
| | | TGCACACGTGCTACAATGGCAGGTACAATGAGCTGCGAAGCCGCGAGGCG |
| | | GAGCGAATCTCAAAAAGCCTGTCTCAGTTCGGATTGGGGTCTGCAACTCGA |
| | | CCCCATGAAGTCGGAGTTGCTAGTAATCGCAGATCAGCATTGCTGCGGTGA |
| | | ATACGTTCCCGGGCCTTGTACACACCGCCCGTCACGTCACGAAAGTCGGTA |
| | | ACACCCGAAGCCGGTGGCCCAACCCCTTGTGGGAGGGAGCTGTCGAAGGT |
| | | GGGACTGGCGATTGGGACGAAGTCGTAACAAGGTAGCCGTACCGGAAGG |
| | | TGCGGCTGGATCACCTCCTTT |

1. A method for conferring a pathogen control or resistance in a plant, the method comprising administering bacteria of a bacterial strain or a microbial active ingredient derived from the bacterial strain to the plant, a part thereof, a seed for growing the plant, or a locus of the plant,
wherein the bacterial strain comprises a 16S polynucleotide having 100% sequence identity to SEQ ID NO: 1, and
wherein the microbial active ingredient is a whole cell broth, a supernatant, an extract, or an extract fraction from a culture of the bacterial strain.

2. (canceled)

3. The method according to claim 1, wherein the pathogen is a bacterium, fungus or is selected from the group consisting of Fusarium spp., Phytophthora spp., Pythium spp., Rhizoctonia spp., Puccinia spp., Mycosphaerella spp., Sclerotinia spp., Botrytis spp., Colletotrichum spp., Microdochium spp., Gaeumannomyces spp., Tapesia spp., Ustilago spp., Ramularia spp., Blumeria spp., Podosphaera spp., Alternaria spp., Monilinia spp., and Penicillium spp; even more preferably wherein the pathogen is Fusarium spp.

4. The method according to claim 1, wherein the the pathogen is a Fusarium spp.

5. The method according to claim 1, wherein the bacterial strain is a Streptomyces bottropensis strain.

6. The method according to claim 1, wherein the bacterial strain is selected from the group consisting of:
the strain deposited under the Budapest Treaty at the Polish Collection of Microorganisms (PCM) on 11 Feb. 2022 under Accession No. B/00366, or a functional mutant thereof;
the strain deposited under the Budapest Treaty at the Polish Collection of Microorganisms (PCM) on 26 Nov. 2021 under Accession No. B/00364, or a functional mutant thereof;
the strain deposited under the Budapest Treaty at the Polish Collection of Microorganisms (PCM) on 2 Nov. 2022 under Accession No. B/00435, or a functional mutant thereof;
the strain deposited under the Budapest Treaty at the Polish Collection of Microorganisms (PCM) on 2 Nov. 2022 under Accession No. B/00436, or a functional mutant thereof;
the strain deposited under the Budapest Treaty at the Polish Collection of Microorganisms (PCM) on 2 Nov. 2022 under Accession No. B/00437, or a functional mutant thereof;
the strain deposited under the Budapest Treaty at the Polish Collection of Microorganisms (PCM) on 2 Nov. 2022 under Accession No. B/00438, or a functional mutant thereof.

7. The method according to claim 1, wherein the bacterial strain is selected from the group consisting of:
the strain deposited under the Budapest Treaty at the Polish Collection of Microorganisms (PCM) on 11 Feb. 2022 under Accession No. B/00366;
the strain deposited under the Budapest Treaty at the Polish Collection of Microorganisms (PCM) on 26 Nov. 2021 under Accession No. B/00364;
the strain deposited under the Budapest Treaty at the Polish Collection of Microorganisms (PCM) on 2 Nov. 2022 under Accession No. B/00435;
the strain deposited under the Budapest Treaty at the Polish Collection of Microorganisms (PCM) on 2 Nov. 2022 under Accession No. B/00436;
the strain deposited under the Budapest Treaty at the Polish Collection of Microorganisms (PCM) on 2 Nov. 2022 under Accession No. B/00437;
the strain deposited under the Budapest Treaty at the Polish Collection of Microorganisms (PCM) on 2 Nov. 2022 under Accession No. B/00438.

8. The method according to claim 1, wherein the bacterial strain is the strain of which a representative culture has been deposited under the Budapest Treaty at the Polish Collection of Microorganisms (PCM) on 11 Feb. 2022 under Accession No. B/00366.

9. The method according to claim 1, the method comprising administering to the plant, the part thereof, the seed for growing the plant, or the locus of the plant:
bacteria of two or more bacterial strains selected from the group consisting of
the strain deposited under the Budapest Treaty at the Polish Collection of Microorganisms (PCM) on 11 Feb. 2022 under Accession No. B/00366, or a functional mutant thereof;
the strain deposited under the Budapest Treaty at the Polish Collection of Microorganisms (PCM) on 26 Nov. 2021 under Accession No. B/00364, or a functional mutant thereof;
the strain deposited under the Budapest Treaty at the Polish Collection of Microorganisms (PCM) on 2 Nov. 2022 under Accession No. B/00435, or a functional mutant thereof;
the strain deposited under the Budapest Treaty at the Polish Collection of Microorganisms (PCM) on 2 Nov. 2022 under Accession No. B/00436, or a functional mutant thereof;
the strain deposited under the Budapest Treaty at the Polish Collection of Microorganisms (PCM) on 2 Nov. 2022 under Accession No. B/00437, or a functional mutant thereof;
the strain deposited under the Budapest Treaty at the Polish Collection of Microorganisms (PCM) on 2 Nov. 2022 under Accession No. B/00438, or a functional mutant thereof, and/or
the two or more bacterial strains in conjunction with one or more additional plant-beneficial microorganism.

10. The method according to claim 1, comprising administering to the plant, the part thereof, the seed for growing the plant, or the locus of the plant:
a microbial active ingredient that is derived from two or more bacterial strains selected from the group consisting of
the strain deposited under the Budapest Treaty at the Polish Collection of Microorganisms (PCM) on 11 Feb. 2022 under Accession No. B/00366, or a functional mutant thereof;
the strain deposited under the Budapest Treaty at the Polish Collection of Microorganisms (PCM) on 26 Nov. 2021 under Accession No. B/00364, or a functional mutant thereof;
the strain deposited under the Budapest Treaty at the Polish Collection of Microorganisms (PCM) on 2 Nov. 2022 under Accession No. B/00435, or a functional mutant thereof;
the strain deposited under the Budapest Treaty at the Polish Collection of Microorganisms (PCM) on 2 Nov. 2022 under Accession No. B/00436, or a functional mutant thereof;
the strain deposited under the Budapest Treaty at the Polish Collection of Microorganisms (PCM) on 2 Nov. 2022 under Accession No. B/00437, or a functional mutant thereof;
the strain deposited under the Budapest Treaty at the Polish Collection of Microorganisms (PCM) on 2 Nov. 2022 under Accession No. B/00438, or a functional mutant thereof, and/or
the microbial active ingredient in conjunction with one or more additional plant-beneficial microorganism,
wherein the microbial active ingredient is a whole cell broth, a supernatant from a culture of the bacterial strain or an extract or extract fraction from a culture of the bacterial strain.

11. The method according to claim 1, wherein the bacteria or the microbial active ingredient are administered in an agricultural active composition, such as wherein:
the composition further comprises one or more agriculturally acceptable auxiliary, such as a solvent, a carrier, a surfactant, a sticker, an antifreeze agent, a thickener, a buffering agent, an antifoaming agent, an antioxidant, a preservative, an aroma, a colorant, or a combination thereof;
the composition is a liquid composition, such as an aqueous composition, such as a sprayable liquid or a concentrate, preferably wherein the composition comprises the bacteria at a concentration of at least about 10² CFU/ml; or
the composition is a non-liquid composition, such as a powder, preferably wherein the composition comprises the bacteria at an amount of at least about 10² CFU/g.

12. An agricultural active composition comprising a bacterial strain or a microbial active ingredient derived from said bacterial strain, wherein the bacterial strain is selected from the group consisting of:
the strain deposited under the Budapest Treaty at the Polish Collection of Microorganisms (PCM) on 11 Feb. 2022 under Accession No. B/00366, or a functional mutant thereof;
the strain deposited under the Budapest Treaty at the Polish Collection of Microorganisms (PCM) on 26 Nov. 2021 under Accession No. B/00364, or a functional mutant thereof;
the strain deposited under the Budapest Treaty at the Polish Collection of Microorganisms (PCM) on 2 Nov. 2022 under Accession No. B/00435, or a functional mutant thereof;
the strain deposited under the Budapest Treaty at the Polish Collection of Microorganisms (PCM) on 2 Nov. 2022 under Accession No. B/00436, or a functional mutant thereof;
the strain deposited under the Budapest Treaty at the Polish Collection of Microorganisms (PCM) on 2 Nov. 2022 under Accession No. B/00437, or a functional mutant thereof; and
the strain deposited under the Budapest Treaty at the Polish Collection of Microorganisms (PCM) on 2 Nov. 2022 under Accession No. B/00438, or a functional mutant thereof; or
wherein the microbial active ingredient is a whole cell broth, a supernatant, an extract or an extract fraction from a culture of the bacterial strain.

13. The agricultural active composition of claim 12, wherein the bacterial strain is selected from the group consisting of:
the strain deposited under the Budapest Treaty at the Polish Collection of Microorganisms (PCM) on 11 Feb. 2022 under Accession No. B/00366;
the strain deposited under the Budapest Treaty at the Polish Collection of Microorganisms (PCM) on 26 Nov. 2021 under Accession No. B/00364
the strain deposited under the Budapest Treaty at the Polish Collection of Microorganisms (PCM) on 2 Nov. 2022 under Accession No. B/00435;
the strain deposited under the Budapest Treaty at the Polish Collection of Microorganisms (PCM) on 2 Nov. 2022 under Accession No. B/00436;
the strain deposited under the Budapest Treaty at the Polish Collection of Microorganisms (PCM) on 2 Nov. 2022 under Accession No. B/00437; and
the strain deposited under the Budapest Treaty at the Polish Collection of Microorganisms (PCM) on 2 Nov. 2022 under Accession No. B/00438.

14. (canceled)

15. The agricultural active composition of claim 12, further comprising one or more agriculturally acceptable auxiliary, a solvent, a carrier, a surfactant, a sticker, an antifreeze agent, a thickener, a buffering agent, an antifoaming agent, an antioxidant, a preservative, an aroma, a colorant, or a combination thereof.

16. The agricultural active composition of claim 12, wherein
the composition is a liquid composition, an aqueous composition, a sprayable liquid or a concentrate; or
the composition is a non-liquid composition, a powder.

17. The agricultural active composition of claim 12 wherein the bacterial strain is present in spray-dried or freeze-dried form.

18. (canceled)

19. A plant or plant part heterologously disposed with the agricultural active composition according to of claim 12.

20. The method according to claim 1, wherein the bacterial strain is the strain of which a representative culture has been deposited under the Budapest Treaty at the Polish Collection of Microorganisms (PCM) on 11 Feb. 2022 under Accession No. B/00364.

21. The agricultural active composition according to claim 12, wherein the composition is a soluble powder, soluble granule, wettable granule, tablet formulation, dry flowable, aqueous flowable, wettable dispersible granule, oil dispersion, suspension concentrate, dispersible concentrate, emulsifiable concentrate, aqueous suspension, fertilizer granule, or a sprayable.

22. A method of administering a composition to a plant, the method comprising administering the composition to the plant, a plant part thereof or a substrate comprising or hosting the plant;
wherein the composition is the agricultural active composition according to claim 12.
